(19) United States
(12) Patent Application Publication
(10) Pub. No.: US 2026/0266796 A1
(43) Pub. Date: Sep. 10, 2026

(54) INFORMATION PROCESSING APPARATUS, INFORMATION PROCESSING METHOD, AND INFORMATION PROCESSING PROGRAM

(71) Applicant: SONY GROUP CORPORATION, TOKYO (JP)

(72) Inventors: TAKAHIRO OISHI, TOKYO (JP); NORIHITO MIHOTA, TOKYO (JP); SACHIO IIDA, TOKYO (JP)

(21) Appl. No.: 18/725,031

(22) PCT Filed: Dec. 26, 2022
(86) PCT No.: PCT/JP2022/047893

§ 371 Date: Jun. 27, 2024

(30) Foreign Application Priority Data
Jan. 5, 2022 (JP) 2022-000629

(51) Int. Cl. G01N 33/18; G01N 33/24; G16Y 10/05; G16Y 20/10; G16Y 40/10

(52) U.S. Cl. CPC G01N 33/1886; G01N 33/246; G16Y 10/05; G16Y 20/10; G16Y 40/10

(57) ABSTRACT

[Object] To place a sensor apparatus at a position that is free from impacts of objects to improve the measurement accuracy.

[Solving Means] An information processing apparatus includes a transmission coefficient calculator that calculates a transmission coefficient from a time waveform of a transmitted wave that is transmitted through the first region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes; and a determination section that determines whether an object is situated in a specified range in the medium with respect to the pair of probes, on the basis of the transmission coefficient. Water spraying on crops can also be controlled according to a result of measurement performed by a moisture sensor. This could result in contributing toward using water more efficiently in the field of agriculture.

### TECHNICAL FIELD

The present disclosure relates to an information processing apparatus, an information processing method, and an information processing program that are used to perform determination regarding a medium such as soil.

### BACKGROUND ART

A soil moisture sensor is an apparatus that measures a moisture amount in a medium such as soil, and is used in the fields of agriculture and of soil environment survey. For example, in the field of agriculture, the use of a soil moisture sensor makes it possible to optimally spray crops with water. The soil moisture sensor is used to enhance added values of products.

### CITATION LIST

### Patent Literature

Patent Literature 1: WO 2018/221051
Patent Literature 2: Japanese Patent Application Laid-open No. 2020-187120
Patent Literature 3: United States Patent Application Publication No. 2018/0224382
Patent Literature 4: Japanese Patent Application Laid-open No. 2014-74601

### DISCLOSURE OF INVENTION

### Technical Problem

There is a need for knowing an appropriate position for placing a soil moisture sensor in use (such as whether the position is free from impacts of extraneous materials, and whether representative values can be obtained at the position), and for measuring a moisture distribution in soil in a direction of depth in the soil. With respect to the soil moisture sensors disclosed in Patent Literatures 1 to 3, when an obstacle (an object) is situated around an antenna of the placed soil moisture sensor, there may be a reduction in measurement accuracy. Existing sensors measure propagation delay times of radio waves between antennas. When an obstacle (an object) is situated around the antenna, an unnecessary propagation path may be created, and this may result in inducing an error in propagation delay time. However, it is difficult for a user to determine whether the sensor is placed properly. Typical moisture sensors perform measurement on the entirety of surroundings of the rods, and thus exhibit low spatial resolutions. This results in difficulty in measuring a moisture distribution in a medium in a direction of depth in the medium. Further, the low spatial resolution makes it possible to reduce impacts of extraneous materials by measurement being performed averagely. On the other hand, when the moisture sensor is placed at a position at which an extraneous material is situated, it will be difficult to completely remove an impact of the extraneous material.

In view of the circumstances described above, it is an object of the present disclosure to improve the accuracy in measurement performed by a soil-moisture sensor apparatus. More specifically, it is an object of the present disclosure to place the sensor apparatus at a position that is free from impacts of obstacles (objects) to improve the measurement accuracy, and to measure a moisture distribution in a medium in a direction of depth in the medium to improve the measurement accuracy.

### Solution to Problem

An information processing apparatus according to an embodiment of the present disclosure includes a transmission coefficient calculator that calculates a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes; and a determination section that determines whether an object is situated in a specified range in the medium with respect to the pair of probes, on the basis of the transmission coefficient.

It can be determined whether an object is situated around the antennas of the placed probes. It can also be used not only to determine the presence of an obstacle but also to positively detect a useful object.

The information processing apparatus may further include a determination result outputting section that outputs, to a display apparatus, a result of the determination performed by the determination section.

This enables a user to know whether there is an impact that an obstacle (an object) has on a measurement result.

When an object has been determined to be situated in the specified range in the medium with respect to the pair of probes, the determination result outputting section may output, to the display apparatus, information indicating that the pair of probes is placed at an inappropriate position in the medium.

This enables a user to place the pair of probes again at a different position.

When an object has been determined to be situated in the specified range in the medium with respect to the pair of probes, the determination result outputting section may output, to the display apparatus, information indicating the specified range in which the object has been determined to be situated.

This enables a user to easily place the pair of probes again outside of the specified range.

When an object has been determined to be situated in the specified range in the medium with respect to the pair of probes, the determination result outputting section may output, to the display apparatus, information indicating a recommendation to place the pair of probes outside of the specified range in the medium.

This enables a user to easily place the pair of probes again in a recommended range.

The information indicating the specified range may include an image that shows the specified range.

A user can easily place the pair of probes again outside of the specified range by viewing the image showing the specified range.

The information processing apparatus may further include an electric-signal-transmission controller that outputs an instruction to the transmitter when a specified trigger occurs, the instruction being used to cause the transmitter to transmit the electric signal.

This enables the transmitter to transmit an electric signal at an appropriate timing.

The specified trigger may include a timing at which the pair of probes is placed, an unchangeable periodic timing, a changeable periodic timing, and/or a change in weather.

The unchangeable periodic timing refers to a timing such as day by day, week by week, or month by month. The changeable periodic timing refers to shortening a period, for example, in bad weather, or in a season in which roots of plants easily grow (frequent occurrence of the timing). The change in weather refers to a state in which stones or rocks in soil are easily movable, for example, in bad weather, in rainy weather, after bad weather is over, or after rainy weather is over.

The determination section may determine whether an object is situated at a specified position in the medium with respect to each of at least the two pairs of probes.

A position of an object can be specified at a point but not in a wide range.

The determination section may include a first determination section. When a signal intensity at a peak for the transmission coefficient that is reached when a value of t is closest to zero exhibits a maximum value in a propagation-time range of between tA and tB, the first determination section determines that no object is situated in a first range that corresponds to the region situated between the probes of the pair of probes, in which tA represents a propagation time of propagation through a first medium that is included in the medium, and tB represents a propagation time of propagation through a second medium that is included in the medium and different from the first medium; and when the signal intensity does not exhibit the maximum value, the first determination section determines that an object is situated in the first range.

It can be determined that an object is situated between the probes of the pair of probes.

The information processing apparatus may further include a propagation time calculator that calculates a propagation time and a signal intensity of the transmitted wave, and calculates a signal intensity A and a time t1m_S21 at a peak of a desired wave. The determination section may include a duration calculator that calculates a duration F of a wave when the wave has a specified signal intensity that is less than the signal intensity A at the peak of the desired wave; and a second determination section that determines, when the duration F is greater than or equal to a first threshold, that an object is situated in a second range extending from the pair of probes, and determines, when the duration F is less than the first threshold, that no object is situated in the second range extending from the pair of probes.

It can be determined whether an object is situated in the second range quite near the pair of probes.

The information processing apparatus may further include a propagation time calculator that calculates a propagation time and a signal intensity of the transmitted wave, and calculates a signal intensity A and a time t_{1m_S21} at a peak of a desired wave. The determination section may include an unnecessary wave calculator that calculates a time difference Δt between the time t_{1m_S21} at the peak of the desired wave and a time t_{2m_S21} at a peak of an unnecessary wave by subtracting the time t_{1m_S21} from the time t_{2m_S21}, the peak of the unnecessary wave being a next peak that is reached after the time t_{1m_S21} at the peak of the desired wave; and a third determination section that determines, when Δt is greater than or equal to a second threshold, that no object is situated in a third range that is larger than the second range, and determines, when Δt is less than the second threshold, that an object is situated in the third range.

It can be determined whether an object is situated in the third range distant from the pair of probes.

The unnecessary wave calculator may calculate a signal intensity B of the unnecessary wave that is obtained when +Δt period of time has elapsed since the time t_{2m_S21} at the peak of the unnecessary wave. When a value obtained by subtracting a value of the signal intensity B from a value of the signal intensity A is greater than or equal to a third threshold, the third determination section may determine that no object is situated in the third range; and when the value obtained by subtracting the value of the signal intensity B from the value of the signal intensity A is less than the third threshold, the third determination section may determine that an object is situated in the third range.

It can be determined whether an object is situated in the third range distant from the pair of probes.

The information processing apparatus may further include a reflection coefficient calculator that calculates a reflection coefficient from a time waveform of a reflected wave that is received by the receiver, the reflected wave being obtained by the incident wave being reflected off the one of the pair of probes; a going-and-returning time calculator that calculates a going-and-returning time and a signal intensity of the reflected wave, and calculates a signal intensity and a time t_{1m_S11} at a peak of the reflected wave; a propagation-delay-time calculator that calculates a propagation delay time t_{pd1} that is a difference between the time t_{1m_S21} at the peak of the desired wave and the time t_{1m_S11} at the peak of the reflected wave, the propagation delay time t_{pd1} being obtained by subtracting the time t_{1m_S11} from the time t_{1m_S21}; and a position calculator that calculates, when the third determination section has determined that an object is situated in the third range, a collection of pieces of position information regarding positions at which the object is likely to be situated, the calculation being performed on the basis of the time difference, a position of a pair of the antennas, relative permittivity of the medium, a light speed, a distance between the probes of the pair of probes, and the propagation delay time t_{pd1}.

When an object is situated in the third range distant from the pair of probes, the collection of pieces of position information regarding positions at which the object may be situated can be calculated.

The information processing apparatus may further include a propagation time calculator that calculates a propagation time and a signal intensity of the transmitted wave, and calculates a signal intensity A and a time t_{1m_S21} at a peak of a desired wave; a reflection coefficient calculator that calculates a reflection coefficient from a time waveform of a reflected wave that is received by the receiver, the reflected wave being obtained by the incident wave being reflected off the one of the pair of probes; a going-and-returning time calculator that calculates a going-and-returning time and a signal intensity of the reflected wave, and calculates a signal intensity and a time t_{1m_S11} at a peak of the reflected wave; a propagation-delay-time calculator that calculates a propagation delay time t_{pd1} that is a difference between the time t_{1m_S21} at the peak of the desired wave and the time t_{1m_S11} at the peak of the reflected wave, the propagation delay time t_{pd1} being obtained by subtracting the time t_{1m_S11} from the time t_{1m_S21}; a moisture amount converter that converts the propagation delay time t_{pd1} into a moisture amount in the medium using a coefficient that represents a relationship between the moisture amount and the propagation delay time, and chronologically records the obtained moisture amounts in a memory; and a moisture amount outputting section that chronologically displays, on a display apparatus, a history of the moisture amounts chronologically recorded in the memory.

The present embodiment makes it possible to measure a moisture distribution in a medium and to present the measured moisture distribution to a user.

Each of the probes of the pair of probes may include a first end and a second end, each first end being connected to the transmitter or the receiver, each second end being spaced from a corresponding one of the first ends in an axial direction that is orthogonal to a spacing direction in which the respective antennas of a pair of the respective antennas are spaced from each other; and each of the respective antennas may be provided to a corresponding one of the pair of probes to be spaced from a corresponding one of the first ends in the axis direction.

The respective antennas are not widely provided in the axial direction of the probes, but are provided at a precise position in the axial direction. Consequently, a region in which the respective antennas measure a moisture amount in the axial direction is small, and the respective antennas have a high spatial resolution in the axial direction. In other words, the respective antennas according to the present embodiment can measure a moisture amount in a precise region in the axial direction, not a moisture amount in the entirety of the medium in the axial direction.

Each of the respective antennas may be provided to a corresponding one of the second ends of the pair of probes.

The respective antennas are respectively provided to the second ends respectively corresponding to the insertion tips of the probes, and this makes it possible to measure a moisture amount in a medium over a wider range in the axial direction when the probes are inserted into and removed from the medium.

The moisture amount converter may continuously obtain the moisture amount in the medium for an insertion-and-removal period of time for which a depth of the respective antennas in the medium keeps on being dynamically changed by the pair of probes being inserted into and removed from the medium in the axial direction, the insertion being performed starting from the side of the second ends; and may chronologically record, in the memory, the moisture amounts obtained for the insertion-and-removal period of time. The moisture amount outputting section may chronologically display, on the display apparatus, the moisture amounts obtained for the insertion-and-removal period of time and chronologically recorded in the memory.

This makes it possible to continuously measure moisture amounts at different positions in a medium in a direction of depth in the medium when the pair of the respective antennas is situated at the different positions in the depth direction. Consequently, moisture amounts continuously measured at different positions in a direction of depth in a medium can be presented to a user as a moisture distribution in the depth direction.

The moisture amount outputting section may display, on the display apparatus, a graph having two axes that respectively represent the history of the moisture amounts chronologically recorded in the memory and an elapsed time corresponding to the chronological order for the recording, or that respectively represent the history of the moisture amounts chronologically recorded in the memory and the chronologically changed depth.

Accordingly, a moisture amount that is contained in a medium and corresponds to an elapsed time for inserting and removing the probes, or the moisture amount being contained in the medium and corresponding to a depth of the respective antennas that is substantially proportional to the elapsed time for inserting and removing the probes, can be displayed in an intuitive manner for a user.

The information processing apparatus may further include an insertion degree converter that converts, into the depth of the respective antennas in the medium, a distance that is measured by a ranging sensor and dynamically changed according to a degree of insertion of the pair of probes, the ranging sensor measuring a distance to a surface of the medium from the ranging sensor; and the moisture amount outputting section may display, on the display apparatus, a graph having two axes that respectively represent the history of the moisture amounts chronologically recorded in the memory and the chronologically changed depth.

The use of the ranging sensor makes it possible to obtain a depth in a medium more accurately.

The moisture amount outputting section may display, on the display apparatus, information that indicates a depth of an object situated in the medium, the depth of the object being estimated on the basis of a change in the moisture amount relative to the depth of the respective antennas in the medium.

A user can take measures such as inserting the probes at another position or placing the probes in a state of slightly protruding from the medium M without being completely inserted, such that the probes are not affected by the object.

Instead of the history of the moisture amounts, the moisture amount outputting section may display, on the graph, a history of desired-wave propagation delay times respectively corresponding to the moisture amounts, the desired-wave propagation delay time being a chronologically changed propagation delay time of the desired wave.

The moisture amount outputting section may further display a history of propagation delay times of an unnecessary wave on the graph.

When the propagation delay time of an unnecessary wave is displayed in addition to the desired-wave propagation delay time, this enables a user to intuitively understand that it is better to change the placement position since an object is situated in the specified range.

The moisture amount outputting section may further display, on the graph, a range in which calculation of the desired-wave propagation delay time is likely to be affected by an object being situated in the specified range in the medium with respect to the pair of probes, the range being determined using the desired-wave propagation delay time as a reference.

When at least a portion of values of the propagation delay time of an unnecessary wave are in the range in which calculation of the desired-wave propagation delay time is likely to be affected, this means that an object may be situated in the specified range in the medium with respect to the pair of probes. When at least a portion of the values of the propagation delay time of an unnecessary wave are in the range in which calculation of the desired-wave propagation delay time is likely to be affected, this enables a user to intuitively understand that it is better to change the placement position since an object is situated in the specified range.

The moisture amount converter may measure the moisture amount frequently during insertion and removal of the pair of probes; and may measure the moisture amount less frequently when the pair of probes is in a non-dynamic state, compared to the case in which the frequent measurement is performed.

This makes it possible to perform optimal measurement according to the dynamic or non-dynamic state of the probes.

The information processing apparatus may further include a fourth determination section that determines whether an object is situated in the specified range in the medium with respect to the pair of probes, on the basis of the history of the moisture amounts chronologically recorded in the memory or on the basis of a history of the propagation delay times and, determines a depth of the object when the object has been determined to be situated in the specified range in the medium with respect to the pair of probes; and a determination result outputting section that outputs information to the display apparatus when the fourth determination section has determined that an object is situated in the specified range in the medium with respect to the pair of probes, the information indicating that the pair of probes is placed at an inappropriate position in the medium.

This enables a user to place the pair of probes again at a different position.

The information processing apparatus may further include a ranging sensor that measures a distance to a surface of the medium from the ranging sensor; and an insertion degree converter that converts, into a depth of the respective antennas in the medium, the distance measured by the ranging sensor and dynamically changed according to a degree of insertion of the pair of probes. The determination result outputting section may output, to the display apparatus, information that indicates a recommendation about the degree of insertion of the pair of probes into the medium, on the basis of the depth of the object and on the basis of the depth of the respective antennas in the medium, the depth of the object being determined by the fourth determination section, the depth of the respective antennas being obtained by the conversion being performed by the insertion degree converter.

For example, the determination result outputting section can make a recommendation for a user about the insertion degree by outputting a message such as “more shallowly” or “more deeply” on the basis of a determination result, since a relationship between the insertion degree and whether placement is appropriate is known.

The information processing apparatus may further include a guide section that outputs, to a sound output apparatus, sound that indicates the information indicating that the pair of probes is placed at an inappropriate position in the medium and/or the information indicating the recommendation about the degree of insertion of the pair of probes into the medium.

The guide section may provide guidance using sound or by performing display on the basis of a determination state or a determination result. Examples of the providing guidance include instructing to perform insertion more slowly when insertion is performed too fast, compared to a determination speed; instructing to change the placement position when an object is situated nearby; making an announcement to change modes; and instructing to follow the mode (such as instructing not to perform movement when the mode is changed to the normal mode, or instructing to perform slow movement when the mode is changed to the high-speed mode).

An information processing apparatus according to an embodiment of the present disclosure includes a transmission coefficient calculator that calculates a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes; a propagation time calculator that calculates a propagation time and a signal intensity of the transmitted wave, and calculates a signal intensity A and a time t_{1m_S21} at a peak of a desired wave; a reflection coefficient calculator that calculates a reflection coefficient from a time waveform of a reflected wave that is received by the receiver, the reflected wave being obtained by the incident wave being reflected off the one of the pair of probes; a going-and-returning time calculator that calculates a going-and-returning time and a signal intensity of the reflected wave, and calculates a signal intensity and a time t_{1m_S11} at a peak of the reflected wave; a propagation-delay-time calculator that calculates a propagation delay time t_{pd1} that is a difference between the time t_{1m_S21} at the peak of the desired wave and the time t_{1m_S11} at the peak of the reflected wave, the propagation delay time t_{pd1} being obtained by subtracting the time t_{1m_S11} from the time t_{1m_S21}; a moisture amount converter that converts the propagation delay time t_{pd1} into a moisture amount in the medium using a coefficient that represents a relationship between the moisture amount and the propagation delay time, and chronologically records the obtained moisture amounts in a memory; and a moisture amount outputting section that chronologically displays, on a display apparatus, a history of the moisture amounts chronologically recorded in the memory.

The present embodiment makes it possible to measure a moisture distribution in a medium and to present the measured moisture distribution to a user.

An information processing method according to an embodiment of the present disclosure includes calculating a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes; and determining whether an object is situated in a specified range in the medium with respect to the pair of probes, on the basis of the transmission coefficient.

An information processing method according to an embodiment of the present disclosure includes calculating a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes; calculating a propagation time and a signal intensity of the transmitted wave; calculating a signal intensity A and a time t_{1m_S21} at a peak of a desired wave; calculating a reflection coefficient from a time waveform of a reflected wave that is received by the receiver, the reflected wave being obtained by the incident wave being reflected off the one of the pair of probes; calculating a going-and-returning time and a signal intensity of the reflected wave; calculating a signal intensity and a time t_{1m_S11} at a peak of the reflected wave; calculating a propagation delay time t_{pd1} that is a difference between the time t_{1m_S21} at the peak of the desired wave and the time t_{1m_S11} at the peak of the reflected wave, the propagation delay time t_{pd1} being obtained by subtracting the time t_{1m_S11} from the time t_{1m_S21}; converting the propagation delay time t_{pd1} into a moisture amount in the medium using a coefficient that represents a relationship between the moisture amount and the propagation delay time; chronologically recording the obtained moisture amounts in a memory; and chronologically displaying, on a display apparatus, a history of the moisture amounts chronologically recorded in the memory.

An information processing program according to an embodiment of the present disclosure causes a processor of an information processing apparatus to operate as a transmission coefficient calculator and a determination section, the transmission coefficient calculator calculating a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes, the determination section determining whether an object is situated in a specified range in the medium with respect to the pair of probes, on the basis of the transmission coefficient.

An information processing program according to an embodiment of the present disclosure causes a processor of an information processing apparatus to operate as a transmission coefficient calculator, a propagation time calculator, a reflection coefficient calculator, a going-and-returning time calculator, a propagation-delay-time calculator, a moisture amount converter, and a moisture amount outputting section, the transmission coefficient calculator calculating a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes, the propagation time calculator calculating a propagation time and a signal intensity of the transmitted wave, the propagation time calculator calculating a signal intensity A and a time t_{1m_S21} at a peak of a desired wave, the reflection coefficient calculator calculating a reflection coefficient from a time waveform of a reflected wave that is received by the receiver, the reflected wave being obtained by the incident wave being reflected off the one of the pair of probes, the going-and-returning time calculator calculating a going-and-returning time and a signal intensity of the reflected wave, the going-and-returning time calculator calculating a signal intensity and a time t_{1m_S11} at a peak of the reflected wave, the propagation-delay-time calculator calculating a propagation delay time t_{pd1} that is a difference between the time t_{1m_S21} at the peak of the desired wave and the time t_{1m_S11} at the peak of the reflected wave, the propagation delay time t_{pd1} being obtained by subtracting the time t_{1m_S11} from the time t_{1m_S21}, the moisture amount converter converting the propagation delay time t_{pd1} into a moisture amount in the medium using a coefficient that represents a relationship between the moisture amount and the propagation delay time, the moisture amount converter chronologically recording the obtained moisture amounts in a memory, the moisture amount outputting section chronologically displaying, on a display apparatus, a history of the moisture amounts chronologically recorded in the memory.

### BRIEF DESCRIPTION OF DRAWINGS

FIG. 1 illustrates an example of a configuration of a measurement apparatus.

FIG. 2 illustrates an example of a configuration of a measurement unit.

FIG. 3 illustrates a functional configuration of an information processing apparatus according to a first embodiment.

FIG. 4 illustrates a flow of an operation of the information processing apparatus.

FIG. 5 is a diagram used to describe occurrence of multipath.

FIG. 6 is a diagram used to describe a time at a peak of a desired wave and a time at a peak of a reflected wave.

FIG. 7 is a diagram used to describe processing performed by an unnecessary wave calculator.

FIG. 8 is a diagram used to describe the time at the peak of a desired wave and a time at a peak of an unnecessary wave.

FIG. 9 is a graph of an example of X with 1 to 9 GHz and a Kaiser window β of 6.0.

FIG. 10 illustrates an xyz coordinate system, with a midpoint between paired antennas being an origin.

FIG. 11 illustrates an example of the surface of an ellipsoid that represents a collection of pieces of position information regarding positions at which the object may be situated.

FIG. 12 illustrates an example of an image that shows a specified range in which an object has been determined to be situated.

FIG. 13 illustrates a functional configuration of an information processing apparatus according to a first modification.

FIG. 14 illustrates a functional configuration of an information processing apparatus according to a second modification.

FIG. 15 illustrates a functional configuration of an information processing apparatus according to a third modification.

FIG. 16 illustrates a functional configuration of an information processing apparatus according to a fourth modification.

FIG. 17 illustrates a functional configuration of an information processing apparatus according to a fifth modification.

FIG. 18 illustrates a functional configuration of an information processing apparatus according to a sixth modification.

FIG. 19 schematically illustrates the measurement apparatus according to the present embodiment, and comparative examples.

FIG. 20 illustrates a functional configuration of an information processing apparatus according to a second embodiment.

FIG. 21 illustrates a flow of an operation of the information processing apparatus.

FIG. 22 illustrates examples of display performed by a moisture amount outputting section.

FIG. 23 schematically illustrates a graph of a moisture amount and a relationship of displacement of a depth of antennas to the graph of the moisture amount according to a first example.

FIG. 24 schematically illustrates a graph of a moisture amount and a relationship of displacement of a depth of antennas to the graph of the moisture amount according to a second example.

FIG. 25 schematically illustrates a graph of a moisture amount and a relationship of displacement of a depth of antennas to the graph of the moisture amount according to a third example.

FIG. 26 schematically illustrates a graph of a moisture amount and a relationship of displacement of a depth of antennas to the graph of the moisture amount according to a fourth example.

FIG. 27 illustrates a moisture sensor according to a comparative example.

FIG. 28 illustrates a functional configuration of an information processing apparatus according to an eighth modification.

FIG. 29 illustrates a flow of an operation of the information processing apparatus.

FIG. 30 schematically illustrates a graph of a moisture amount and a relationship of displacement of a depth of antennas to the graph of the moisture amount.

FIG. 31 illustrates a ninth modification.

FIG. 32 illustrates a tenth modification.

FIG. 33 illustrates a flow of an operation according to an eleventh modification.

FIG. 34 illustrates a twelfth modification.

FIG. 35 illustrates a functional configuration of an information processing apparatus according to a thirteenth modification.

FIG. 36 illustrates a flow of an operation of the information processing apparatus.

FIG. 37 illustrates a functional configuration of an information processing apparatus according to a fourteenth modification.

FIG. 38 illustrates a functional configuration of an information processing apparatus according to a fifteenth modification.

FIG. 39 illustrates a functional configuration of an information processing apparatus according to a sixteenth modification.

FIG. 40 illustrates a flow of an operation of the information processing apparatus.

### MODE(S) FOR CARRYING OUT THE INVENTION

Embodiments according to the present disclosure will now be described below with reference to the drawings.

### I. First Embodiment

### 1. Example of Configuration of Measurement Apparatus

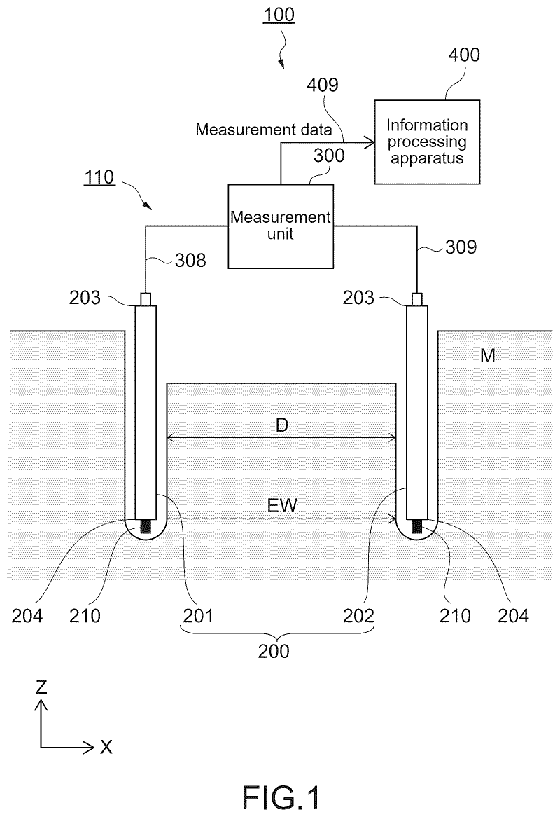

FIG. 1 illustrates an example of a configuration of a measurement apparatus.

A measurement apparatus 100 measures a moisture amount in a medium M, and includes a sensor apparatus 110. For example, soil where crops will grow is assumed to be the medium M.

The sensor apparatus 110 acquires, as measurement data, data necessary to measure a moisture amount. The sensor apparatus 110 transmits the measurement data to an information processing apparatus 400 using a signal 409.

The information processing apparatus 400 can communicate with the sensor apparatus 110, and is, for example, a terminal apparatus (such as a smartphone, a tablet computer, or a personal computer) that is used by an end user (such as a farmer or a farmwork manager). In this case, the signal 409 is carried from the sensor apparatus 110 to the information processing apparatus 400 by wireless communication. Alternatively, the information processing apparatus 400 may be built in a body of the sensor apparatus 110. In this case, the signal 409 may be carried from the sensor apparatus 110 to the information processing apparatus 400 by wired communication or wireless communication. The information processing apparatus 400 measures a moisture amount using measurement data.

The sensor apparatus 110 includes a sensor head 200 and a measurement unit 300. The sensor head 200 is a component including a pair of probes 201 and 202. The probes 201 and 202 are connected to the measurement unit 300 using cables and/or wires 308 and 309 on a substrate. For example, coaxial cables may be used as the cables and/or the wires 308 and 309 on the substrate. The cables and/or the wires 308 and 309 on the substrate are connected to the probes 201 and 202 by tips of the cables and/or the wires 308 and 309 on the substrate being embedded in the respective probes 201 and 202. The measurement unit 300 causes an electromagnetic wave EW to be transmitted by one of the probes 201 and 202 and causes the electromagnetic wave EW to be received by another of the probes 201 and 202. Accordingly, the measurement unit 300 generates measurement data.

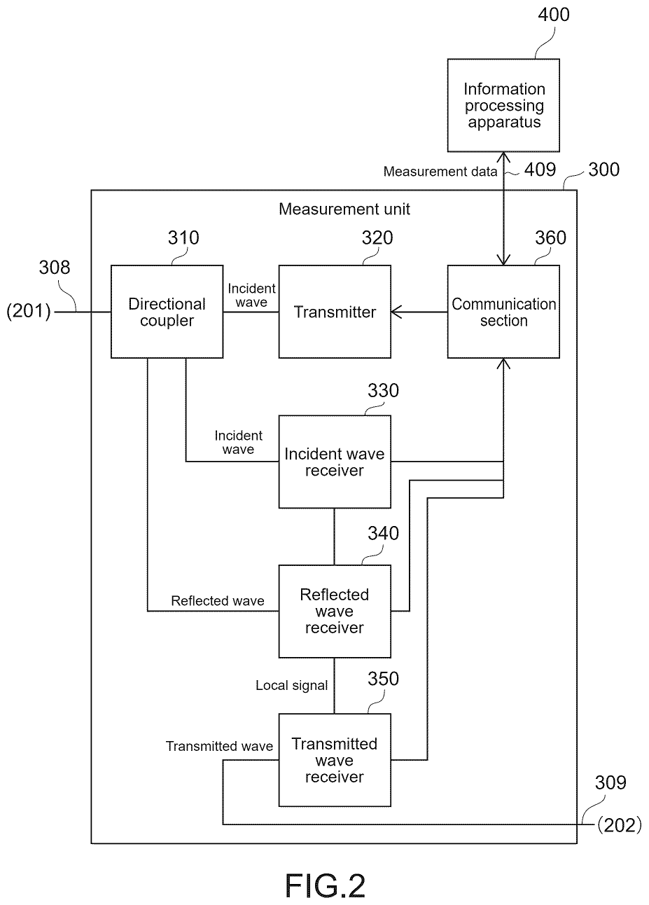

The sensor head 200 includes the probes 201 and 202. The probes 201 and 202 each have a length of, for example, from 75 to 150 millimeters (mm). The probes 201 and 202 each have a thickness (a diameter or a width of a probe's cross section) of, for example, from 3 to 30 millimeters (mm). The probes 201 and 202 are arranged in a medium such as soil, and respectively include antennas 210 that enable an electromagnetic wave of a specified frequency to be transmitted and received between the probes 201 and 202. The paired probes 201 and 202 each have a shape of an elongated rod, and each include a first end 203 and a second end 204. The respective first ends 203 of the probes 201 and 202 are each connected to a transmitter 320 or a transmitted wave receiver 350 in the measurement unit 300 (FIG. 2). The respective second ends 204 of the probes 201 and 202 are respectively spaced from the respective first ends 203 of the probes 201 and 202 in an axial direction Z of axes of the probes 201 and 202, and are tips to be inserted into the medium M. In other words, the axial direction Z of the probes 201 and 202 is an insertion direction in which the probes 201 and 202 are inserted into the medium M. The axial direction Z of the probes 201 and 202 is a direction that is orthogonal to a spacing direction X in which the paired antennas 210 are spaced from each other. The respective antennas 210 of the probes 201 and 202 are provided to be spaced from the respective first ends 203 of the probes 201 and 202 in the axial direction Z. Specifically, the respective antennas 210 of the probes 201 and 202 are respectively provided at the respective second ends 204 of the probes 201 and 202. In other words, the respective antennas 210 are not widely provided in the axial direction Z of the probes 201 and 202, but are provided at a precise position in the axial direction Z. Consequently, a region in which the respective antennas 210 measure a moisture amount in the axial direction Z is smaller, and the respective antennas 210 have a higher spatial resolution in the axial direction Z, compared to when antennas are provided widely in the axial direction Z of axes of probes.

The probes 201 and 202 are embedded in a medium such that a distance between the respective antennas 210 of the probes 201 and 202 in the spacing direction X exhibits a specified value D. For example, the probes 201 and 202 are embedded in the medium M in a generally vertical pose in the axial direction Z. Note that the pose of the probes 201 and 202 is not limited to the vertical pose if the distance between the respective antennas 210 exhibits the value D. Further, it is conceivable that a vertical hole could be dug in the medium M in, for example, a fruit farm using an excavator such as a shovel, and that the probes 201 and 202 could be inserted into the medium M from an inner circumferential wall surface of the vertical hole. In this case, the axial direction Z of the probes 201 and 202 (that is, a direction of depth upon insertion) is not a vertical direction (a longitudinal direction), but is a horizontal direction (a lateral direction). The present embodiment can also be applied to such a use case.

The magnitude of the distance D between the antennas 210 is not particularly limited. If the distance D is too large, there may be an increase in the attenuation of the electromagnetic wave EW propagating through the medium M, which may result in difficulty in obtaining a sufficient reception intensity. On the other hand, if the distance D is too small, there may be a technical difficulty in performing observation. In consideration of the matter described above, the distance D is set to an appropriate value. For example, the distance D is from 25 to 75 millimeters (mm).

### 2. Measurement Unit

FIG. 2 illustrates an example of a configuration of the measurement unit.

The measurement unit 300 includes a directional coupler 310, the transmitter 320, a communication section 360, an incident wave receiver 330, a reflected wave receiver 340, and the transmitted wave receiver 350. For example, a vector network analyzer is used as the measurement unit 300.

The directional coupler 310 splits, into an incident wave and a reflected wave, an electric signal carried by the cable and/or the wire 308 on the substrate. The incident wave is a wave of an electric signal transmitted by the transmitter 320, and the reflected wave is a wave obtained by the incident wave being reflected off an end of the probe 201. The directional coupler 310 supplies the incident wave to the incident wave receiver 330, and supplies the reflected wave to the reflected wave receiver 340.

The transmitter 320 transmits, as an electric signal, the electric signal of a specified frequency to the probe 201 using the directional coupler 310 and using the cable and/or the wire 308 on the substrate. For example, a continuous wave (CW wave) is used as an incident wave included in the electric signal. The transmitter 320 transmits an electric signal while successively switching frequencies in steps of 50 megahertz (MHz) in a range of frequencies of, for example, from one to nine gigahertz (GHz).

The incident wave receiver 330 receives an incident wave from the directional coupler 310. The reflected wave receiver 340 receives a reflected wave from the directional coupler 310. The transmitted wave receiver 350 receives a transmitted wave from the probe 202. Here, the transmitted wave is obtained by the probe 202 converting, into an electric signal, an electromagnetic wave transmitted through a region, in the medium, that is situated between the probes 201 and 202.

The incident wave receiver 330, the reflected wave receiver 340, and the transmitted wave receiver 350 perform quadrature detection and analog-to-digital (AD) conversion with respect to the respectively received incident wave, reflected wave, and transmitted wave, and transmit respective pieces of obtained information to the information processing apparatus 400 as pieces of measurement data using the signal 409.

### 3. Functional Configuration of Information Processing Apparatus

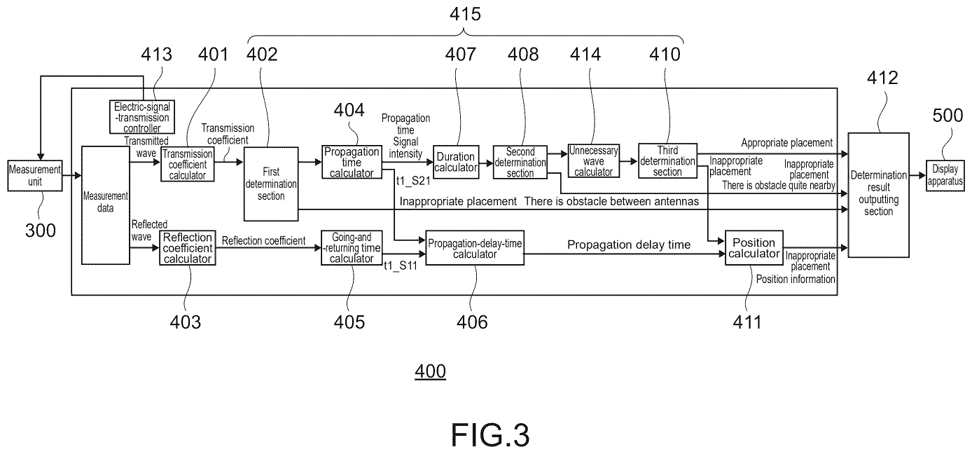

FIG. 3 illustrates a functional configuration of the information processing apparatus according to the first embodiment.

The information processing apparatus 400 operates as an electric-signal-transmission controller 413, a transmission coefficient calculator 401, a first determination section 402, a reflection coefficient calculator 403, a propagation time calculator 404, a going-and-returning time calculator 405, a propagation-delay-time calculator 406, a duration calculator 407, a second determination section 408, an unnecessary wave calculator 414, a third determination section 410, a position calculator 411, and a determination result outputting section 412 by a processor such as a CPU loading, into a RAM, an information processing program stored in a ROM and executing the information processing program.

The first determination section 402, the duration calculator 407, the second determination section 408, the unnecessary wave calculator 414, and the third determination section 410 are included in a determination section 415. The determination section 415 determines whether an object is situated in a specified range in a medium with respect to the pair of probes 201 and 202.

The information processing apparatus 400 further includes a moisture amount converter (described in a second embodiment). The moisture amount converter measures a moisture amount on the basis of a going-and-returning time (described later), a propagation time (described later), and a propagation delay time (described later). First, the moisture amount converter calculates a propagation delay time from a going-and-returning time and a propagation time. The moisture amount converter reads a coefficient that represents a relationship between a moisture amount and a propagation delay time. The moisture amount converter converts the calculated propagation delay time into a moisture amount using the coefficient. The moisture amount converter outputs the obtained moisture amount to an external apparatus or external equipment as necessary.

### 4. Information Processing Apparatus

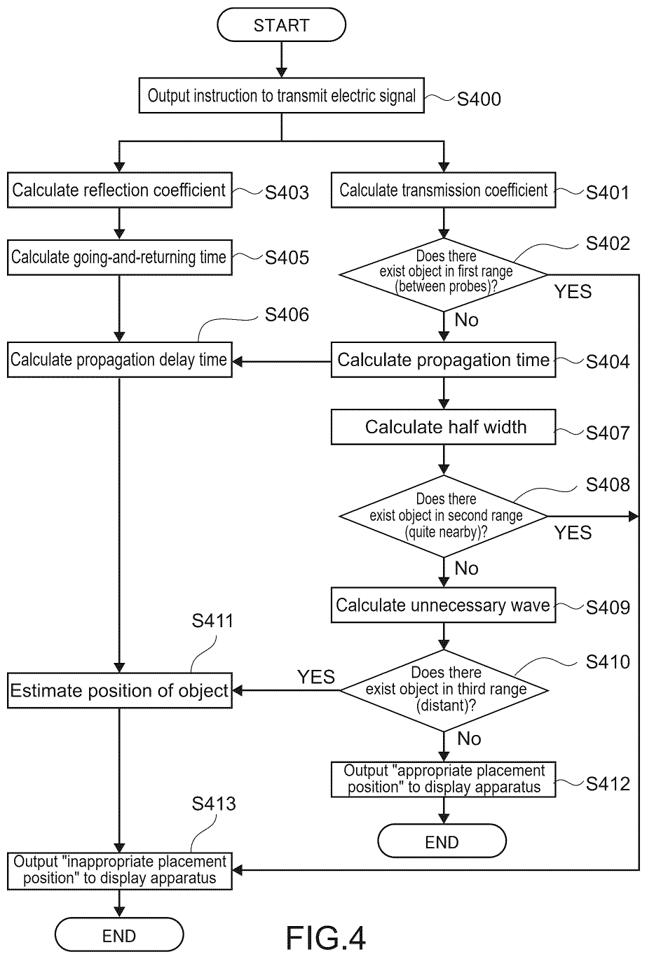

FIG. 4 illustrates a flow of an operation of the information processing apparatus.

When a specified trigger occurs, the electric-signal-transmission controller 413 outputs, to the transmitter 320 of the measurement unit 300, an instruction that is used to cause the transmitter 320 to transmit an electric signal including an incident wave (Step S400). The specified trigger includes a timing at which the pair of probes 201 and 202 is placed, an unchangeable periodic timing, a changeable periodic timing, and/or a change in weather. The unchangeable periodic timing refers to a timing such as day by day, week by week, or month by month. The changeable periodic timing refers to shortening a period, for example, in bad weather, or in a season in which roots of plants easily grow (frequent occurrence of the timing). The change in weather refers to a state in which stones or rocks in soil are easily movable, for example, in bad weather, in rainy weather, after bad weather is over, or after rainy weather is over. In response to receiving the instruction, the transmitter 320 transmits an electric signal including an incident wave.

The transmission coefficient calculator 401 calculates a transmission coefficient from a time waveform of a transmitted wave received by the transmitted wave receiver 350 (Step S401).

The first determination section 402 determines a signal intensity at a peak for the transmission coefficient that is reached when a value of t is closest to zero in a propagation-time range of between tA and tB (Step S402). tA represents a propagation time of propagation through a first medium (such as air) that is included in the medium. tB represents a propagation time of propagation through a second medium (such as water) that is included in the medium and different from the first medium. When the signal intensity at the peak for the transmission coefficient that is reached when the value of t is closest to zero exhibits a maximum value between tA and tB, the first determination section 402 determines that this peak is a peak of a desired wave and that no object is situated in a first range that corresponds to a region situated between the paired probes 201 and 202 (NO in Step S402). On the other hand, when the signal intensity at the peak for the transmission coefficient that is reached when the value of t is closest to zero does not exhibit a maximum value between tA and tB, the first determination section 402 determines that the pair of probes 201 and 202 is placed at an inappropriate position (YES in Step S402) since an obstacle (an object) may be situated in the first range corresponding to the region situated between the paired probes 201 and 202. Examples of the obstacle (the object) include a root of or branch of a plant, a stone, and a rock.

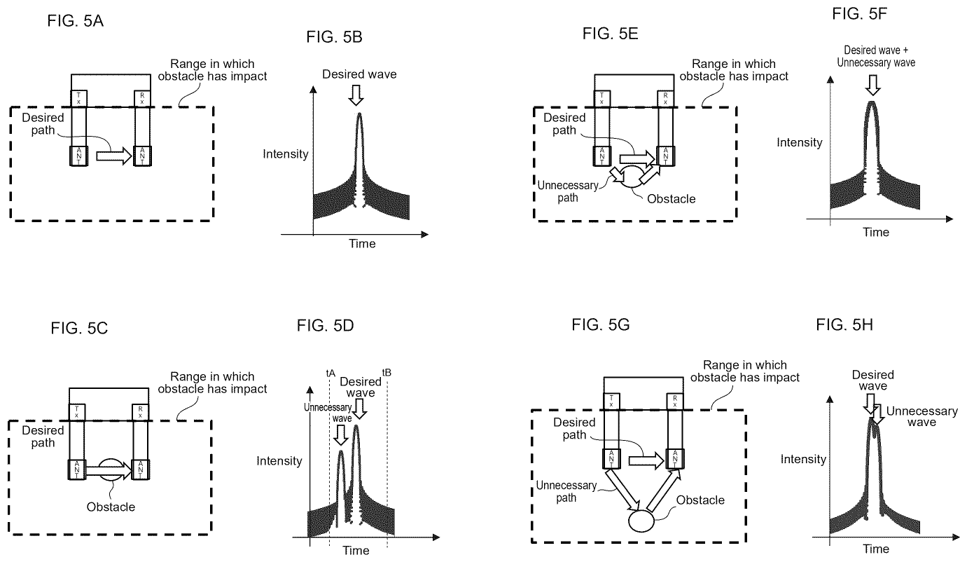

FIG. 5 is a diagram used to describe occurrence of multipath (two paths that respectively correspond to a desired wave and un unnecessary wave).

When no obstacle (no object) is situated in the first range corresponding to the region situated between the paired probes 201 and 202 (NO in Step S402), as in the case of (A), a desired wave that represents a propagation time of propagation through the region between the paired probes 201 and 202, exists in a temporal-axis waveform (B).

On the other hand, when an obstacle (an object) is situated in the first range corresponding to the region situated between the paired probes 201 and 202 (YES in Step S402), as in the case of (C), a wave reflected off the obstacle (the object) is an unnecessary path (the multipath occurs), and an unnecessary wave is caused in the temporal-axis waveform (D). Specifically, the signal intensity at the peak reached when the value of t is closest to zero does not exhibit a maximum value between the times tA and tB.

On the other hand, the reflection coefficient calculator 403 calculates a reflection coefficient from a time waveform of a reflected wave received by the reflected wave receiver 340 (a reflected wave obtained by an incident wave being reflected off the probe 201) (Step S403).

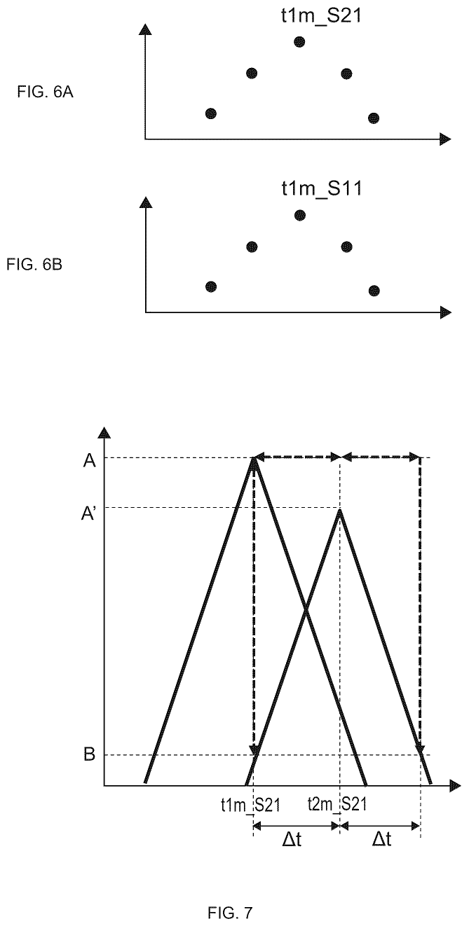

FIG. 6 is a diagram used to describe a time at a peak of a desired wave and a time at a peak of a reflected wave.

When the first determination section 402 has determined that no object is situated in the first range corresponding to the region situated between the paired probes 201 and 202 (NO in Step S402), the propagation time calculator 404 calculates a propagation time and a signal intensity of a transmitted wave, and calculates a signal intensity A and a time t_{1m_S21} at a peak of a desired wave (Step S404). With respect to the desired wave's peak at which a propagation time and a corresponding signal intensity are given, a time at which the signal intensity exhibits a maximum value is set to a time t_{1m_S21}, and the desired wave's peak is obtained using signal intensities at several points at times before and after the time t_{1m_S21}, as illustrated in (A) of FIG. 6. For example, fitting is performed using a quadratic function, and a time corresponding to a point at which the maximum value is exhibited, is set to the time t_{1m_S21}. Fitting may be performed using an approach other than the quadratic function. Further, setting may be performed such that t_{1m_S21}=t_{1_S21}, without performing fitting.

The going-and-returning time calculator 405 calculates a going-and-returning time and a signal intensity of a reflected wave, and calculates a signal intensity and a time t_{1m_S11} at a peak of the reflected wave (Step S405). With respect to the desired wave's peak at which a going-and-returning time and a corresponding signal intensity are given, a time at which the signal intensity exhibits a maximum value is set to a time t_{1_S11}, and the desired wave's peak is obtained using signal intensities at several points at times before and after the time t_{1_S11}, as illustrated in (B) of FIG. 6. For example, fitting is performed using a quadratic function, and a time corresponding to a point at which the maximum value is exhibited, is set to the time t_{1m_S11}. Fitting may be performed using an approach other than the quadratic function. Further, setting may be performed such that t_{1m_S11}=t_{1_S11}, without performing fitting.

The propagation-delay-time calculator 406 calculates a propagation delay time t_{pd1} that is a difference between the time t_{1m_S21} at the peak of a desired wave and the time t_{1m_S11} at the peak of a reflected wave, the propagation delay time t_{pd1} being obtained by subtracting the time t_{1m_S11} from the time t_{1m_S21} (Step S406). The propagation delay time is a time for which an electromagnetic wave propagates through a region, in the medium, that is situated between the probes 201 and 202.

The duration calculator 407 calculates a duration F of a wave when the wave has a specified signal intensity that is less than the signal intensity A (the signal intensity A obtained in Step S404) at the peak of a desired wave (Step S407) The specified signal intensity that is less than the signal intensity A at the peak of the desired wave refers to, for example, a signal intensity that is less than or equal to half the signal intensity A at the peak of the desired wave. The duration F of a wave when the wave has the specified signal intensity may be hereinafter referred to as a half width F. For example, when the signal intensity A at the peak of the desired wave is 6 dB, the signal intensity less than or equal to the signal intensity A is less than or equal to 3 dB. F does not necessarily have to represent a half width, and may represent a duration when the signal intensity is X dB or more lower than the signal intensity at the peak of the desired wave.

The second determination section 408 determines whether the duration F is greater than or equal to a first threshold to determine whether an object is situated in a second range (Step S408). Specifically, the second range is a range that is situated quite near at least one of the pair of probes 201 and 202, as illustrated in (E) of FIG. 5. When the duration F is greater than or equal to a first threshold, the second determination section 408 determines that the pair of probes 201 and 202 is placed at an inappropriate position (YES in Step S408) since an object may be situated in the second range extending from the pair of probes 201 and 202. “The duration F is greater than or equal to the first threshold” means that two peaks overlap and the half width is large, as illustrated in (F) of FIG. 5. On the other hand, when the duration F is less than the first threshold, the second determination section 408 determines that no object is situated in the second range extending from the pair of probes 201 and 202 (NO in Step S408).

FIG. 7 is a diagram used to describe processing performed by an unnecessary wave calculator.

The unnecessary wave calculator 414 calculates a time difference Δt between the time t_{1m_S21} at the peak of a desired wave and a time t_{2m_S21} at a peak of an unnecessary wave by subtracting the time t_{1m_S21} from the time t_{2m_S21}, the peak of the unnecessary wave being a next peak that is reached after the time t_{1m_S21} at the peak of the desired wave. Further, the unnecessary wave calculator 414 calculates a signal intensity B of the unnecessary wave that is obtained when +Δt period of time has elapsed since the time t_{2m_S21} at the peak of the unnecessary wave (Step S409).

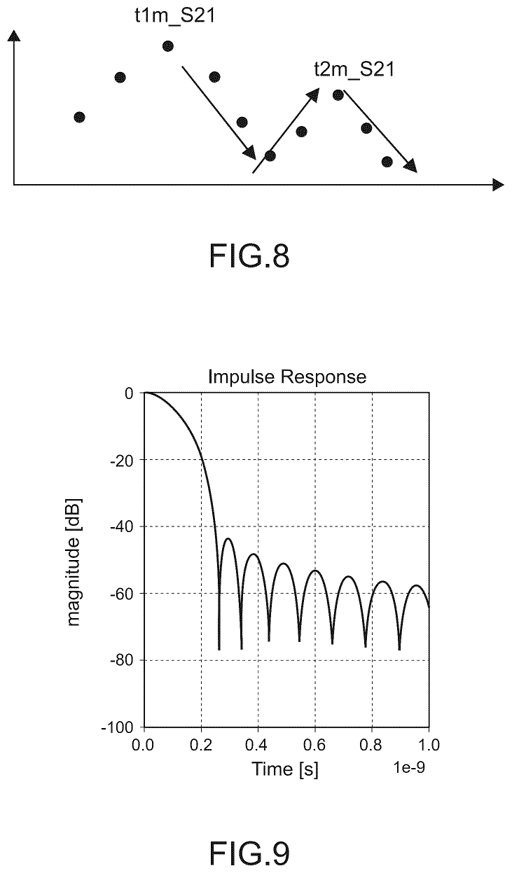

FIG. 8 is a diagram used to describe the time at the peak of a desired wave and the time at the peak of an unnecessary wave.

Specifically, the unnecessary wave calculator 414 checks a signal intensity in a positive direction on a temporal axis after the time t_{1m_S21} at the peak of a desired wave (that is, a signal intensity of a transmitted wave at a time after the time t_{1m_S21}). The unnecessary wave calculator 414 sets, to the time t_{2m_S21} at the peak of an unnecessary wave, a time at which the second maximum value is exhibited, the time being in an amplitude including an increase in signal intensity from a previous amplitude and a decrease in signal intensity after the increase. The signal intensity at that time is referred to as a signal intensity A′.

For example, fitting is performed using a quadratic function, and a time corresponding to a point at which the maximum value is exhibited, is set to the time t_{2m_S21}. Fitting may be performed using an approach other than the quadratic function. Further, setting may be performed such that t_{2m_S21}=t_{2_S21}, without performing fitting.

Next, the unnecessary wave calculator 414 calculates the signal intensity B of the unnecessary wave, the signal intensity B of the unnecessary wave being obtained when +Δt period of time has elapsed since the peak of the unnecessary wave. The signal intensity B is calculated using a function g(T) (T represents a time) of an envelope of the unnecessary wave. B is obtained when g(Δt) is known. The function g(T,win,Fr) differs depending on a window function and a swept frequency range. When, for example, a certain swept frequency range is Fr, a window function is win, and a fast inverse Fourier transform performed in the frequency range Fr is IFFT, there are the following relationships: g(T)=X(T), X=|IFFT(win)|. g(T) is dependent on the window function and the swept frequency range. Examples of the window function include a Kaiser window and a Hamming window. The window function does not necessarily have to be used.

FIG. 9 is a graph of an example of X with 1 to 9 GHz and a Kaiser window β of 6.0.

Thus, the signal intensity B [dB] is represented by a formula indicated below.

$$B = {A}^{′} + g ( Δ t ) [ dB ]$$

The third determination section 410 determines an impact of an unnecessary wave using a value obtained by subtracting a value of the signal intensity B from a value of the signal intensity A and using Δt to determine whether an object is situated in a third range (Step S410). Specifically, the third range is a range that is distant from the pair of probes 201 and 202 and larger than the second range, as illustrated in (G) of FIG. 5.

As an example, when Δt is greater than or equal to a second threshold (for example, 500 ps), the third determination section 410 determines that no object is situated in the third range and determines that the pair of probes 201 and 202 is placed at an appropriate position (NO in Step S410). On the other hand, when Δt is less than the second threshold, the third determination section 410 determines that the pair of probes 201 and 202 is placed at an inappropriate position (YES in Step S410) since an object may be situated in the third range. “At is less than the second threshold” means that, when an unnecessary wave is temporally close to a desired wave, the desired wave will get out of shape, and the position of the peak of the desired wave will be shifted to reduce the measurement accuracy, as illustrated in (H) of FIG. 5.

As another example, when the value obtained by subtracting the value of the signal intensity B from the value of the signal intensity A is greater than or equal to a third threshold (for example, 20 dB), the third determination section 410 determines that no object is situated in the third range and determines that the pair of probes 201 and 202 is placed at an appropriate position (NO in Step S410). On the other hand, when the value obtained by subtracting the value of the signal intensity B from the value of the signal intensity A is less than the third threshold, the third determination section 410 determines that the pair of probes 201 and 202 is placed at an inappropriate position (YES in Step S410) since an object may be situated in the third range.

When the third determination section 410 has determined that an object is situated in the third range (YES in Step S410), the position calculator 411 calculates a collection of pieces of position information regarding positions at which the object may be situated, the calculation being performed on the basis of a time difference Δt, a position of the pair of respective antennas 210, relative permittivity of a medium, a light speed, a distance between the paired probes 201 and 202, and the propagation delay time t_{pd1} to estimate a position of an obstacle (the object) (Step S411). Specifically, the collection of pieces of position information regarding positions at which the object may be situated refers to the surface of an ellipsoid centered at the midpoint between the paired respective antennas 210.

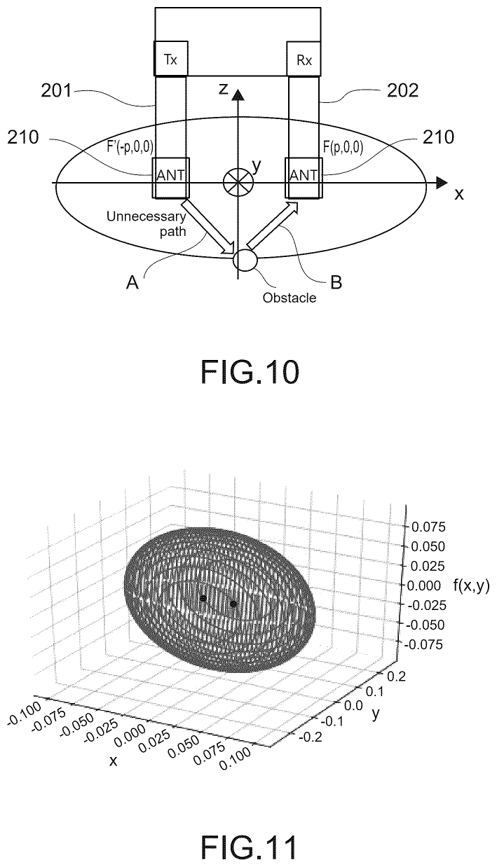

FIG. 10 illustrates an xyz coordinate system, with a midpoint between paired antennas being an origin.

Specifically, the position calculator 411 creates an xyz coordinate system as illustrated in FIG. 10, with the midpoint between the paired respective antennas 210 being an origin. A pair of positional coordinates of an obstacle (an object) is assumed to be represented by (x,y,z), and pairs of positional coordinates of the paired respective antennas 210 are assumed to be respectively represented by F(p,0,0) and F′(−p,0,0).

A distance 1 of an unnecessary path is indicated by arrows A and B in the figure, and is represented by Mathematical Formula 1.

$$l = \sqrt{{( x + p )}^{2} + {y}^{2} + {z}^{2}} + \sqrt{{( x - p )}^{2} + {y}^{2} + {z}^{2}} \qquad [ Math . 1 ]$$

First, when the relative permittivity of a medium is represented by εr, a light speed is represented by c, a distance between probes is represented by L, the propagation delay time t_{pd1} is obtained using the following formula: t_{pd1}=t_{1m_S21}−t_{1m_S11}, √εr is represented by Mathematical Formula 2.

$$\sqrt{ε r} = \frac{{ct}_{pd 1}}{L} \qquad [ Math . 2 ]$$

l is represented by Mathematical Formula 3 using a propagation delay time t_{pd2} of the unnecessary path that is obtained using the following formula: t_{pd2}=t_{2m_S21}−t_{1m_S11}.

$$l = \frac{{ct}_{pd 2}}{\sqrt{ε r}} = \frac{{Lt}_{pd 2}}{{t}_{pd 1}} \qquad [ Math . 3 ]$$

Here, t_{pd2}=Δt+t_{1m_S21}−t_{1m_S11}=Δt+t_{pd1} since Δt=t_{2m_S21}−t_{1m_S21}. This results in obtaining Mathematical Formula 4.

$$l = \frac{L ( Δ t + {t}_{pd 1} )}{{t}_{pd 1}} \qquad [ Math . 4 ]$$

Thus, a solution that provides a position of an obstacle (an object) exists on an ellipsoid function that satisfies Mathematical Formula 5.

$$\frac{L ( Δ t + {t}_{pd 1} )}{{t}_{pd 1}} = \sqrt{{( x + p )}^{2} + {y}^{2} + {z}^{2}} + \sqrt{{( x - p )}^{2} + {y}^{2} + {z}^{2}} \qquad [ Math . 5 ]$$

Here, Mathematical Formula 5 is represented by a, as indicated in Mathematical Formula 6.

$$\frac{L ( Δ t + {t}_{pd 1} )}{{t}_{pd 1}} = α \qquad [ Math . 6 ]$$

In this case, the solution providing the position of the obstacle (the object) exists on an ellipsoid function that satisfies Mathematical Formula 7.

$$\frac{4 ( {α}^{2} - 4 {p}^{2} )}{{α}^{4} - 4 {α}^{2} {p}^{2}} {x}^{2} + \frac{4 {α}^{2}}{{α}^{4} - 4 {α}^{2} {p}^{2}} {y}^{2} + \frac{4 {α}^{2}}{{α}^{4} - 4 {α}^{2} {p}^{2}} {z}^{2} = 1 \qquad [ Math . 7 ]$$

FIG. 11 illustrates an example of the surface of an ellipsoid that represents the collection of pieces of position information regarding positions at which the object may be situated.

In the figure, two dots respectively represent the positions (−p,0,0) and (p,0,0) of the paired respective antennas 210. The distance L between probes=30e-3 [m], Δt=2000e-12 [s], the propagation delay time t_{pd1}=400e-12 [s], and p=L/2 [m]. Δt represents a time difference between a time at a peak of a desired wave and a time at a peak of an unnecessary wave, the peak of the unnecessary wave being a next peak that is reached after the time at the peak of the desired wave.

When the third determination section 410 has determined that no object is situated in the third range and determined that the pair of probes 201 and 202 is placed at an appropriate position (NO in Step S410), the determination result outputting section 412 outputs, to a display apparatus 500, information indicating that the pair of probes 201 and 202 is placed at an appropriate position in a medium (Step S412). Examples of the display apparatus 500 include a display of the information processing apparatus 400 and an LED light. The information processing apparatus 400 may further include a sound output apparatus and an oscillation apparatus. The display apparatus 500, the sound output apparatus, and the oscillation apparatus may be apparatuses that are separate from the information processing apparatus 400 and can communicate with the information processing apparatus 400. For example, the display apparatus 500 may be a transmissive head-mounted display. In this case, communication may be performed using a communication approach such as Bluetooth (registered trademark) or LTE (registered trademark).

On the other hand, the case in which it has been determined that the pair of probes 201 and 202 is placed at an inappropriate position since an obstacle (an object) may be situated in the first range corresponding to the region situated between the paired probes 201 and 202 (YES in Step S402), since an object may be situated in the second range corresponding to a quite nearby region (YES in Step S408), or since an object may be situated in the third range corresponding to a distant region (YES in Step S410) is described. In this case, the determination result outputting section 412 outputs, to the display apparatus 500, information (such as a message) indicating that the pair of probes 201 and 202 is currently placed at an inappropriate position in the medium (Step S413).

Further, the determination result outputting section 412 may output, to the display apparatus 500, information indicating a specified range in which an object has been determined to be situated, or a rough distance to the specified range. This enables a user to easily place the pair of probes 201 and 202 again outside of the specified range. The information indicating the specified range may include an image that shows the specified range (the first range corresponding to the region situated between paired probes, the second range corresponding to a quite nearby range, or an ellipsoid).

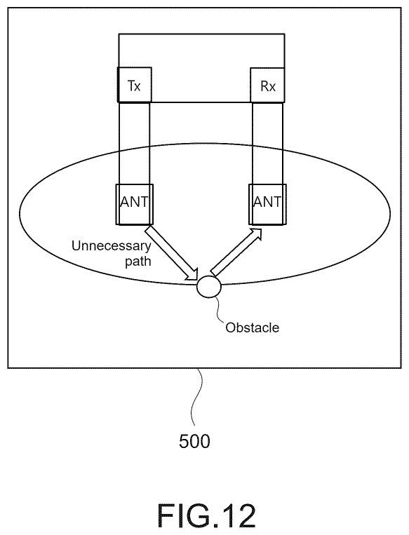

FIG. 12 illustrates an example of the image showing the specified range in which an object has been determined to be situated.

As illustrated in the figure, objects that represent the pair of probes 201 and 202 as viewed from a horizontal direction and the specified range as viewed from the horizontal direction may be in the image showing the specified range, where the specified range as viewed from the horizontal direction is a locus of an ellipsoid in this example, and may be the first range corresponding to the region situated between the paired probes, or the second range corresponding to a quite nearby region. Alternatively, objects that represent the pair of probes 201 and 202 as viewed from a vertical direction and the specified range as viewed from the horizontal direction may be in the image showing the specified range, where the specified range as viewed from the horizontal direction may be the first range corresponding to the region situated between the paired probes, the second range corresponding to a quite nearby region, or an ellipsoid. The image showing the specified range may be superimposed to be displayed on a map of a location at which the pair of probes 201 and 202 is placed. For example, the map may be acquired via the Internet on the basis of position information regarding a position of the information processing apparatus 400, the position information being acquired by the information processing apparatus 400 using a GPS receiving unit. The map may be a two-dimensional or three-dimensional map. Alternatively, when the display apparatus 500 is a transmissive head-mounted display, the image showing the specified range may be superimposed to be displayed on the transmissive head-mounted display in augmented reality (AR) or virtual reality (VR). This enables a user to easily place the pair of probes 201 and 202 again outside of the specified range.

Further, the determination result outputting section 412 may output, to the display apparatus 500, information indicating a recommendation to place the pair of probes 201 and 202 outside of the specified range (the first range corresponding to the region situated between the paired probes, the second range corresponding to a quite nearby region, or an ellipsoid) in the medium. The information indicating the recommendation may be a message and/or an image. Objects that represent the pair of probes 201 and 202 and a recommended placement position may be in the image. As in the case described above, the objects may be superimposed on a map, or may be superimposed to be displayed on a transmissive head-mounted display in augmented reality (AR) or virtual reality (VR). This enables a user to easily place the pair of probes 201 and 202 again in a recommended range.

Further, when the paired probes 201 and 202 are respectively placed at a plurality of different placement locations, the determination result outputting section 412 may simultaneously display information (an image) indicating the specified range in which an object has been determined to be situated, and a placement position to be recommended (an image), on the basis of the plurality of different placement locations. As in the case described above, the objects may be superimposed on a map, or may be superimposed to be displayed on a transmissive head-mounted display in augmented reality (AR) or virtual reality (VR). This enables a user to easily place the pair of probes 201 and 202 again outside of the specified range.

### 5. Modifications

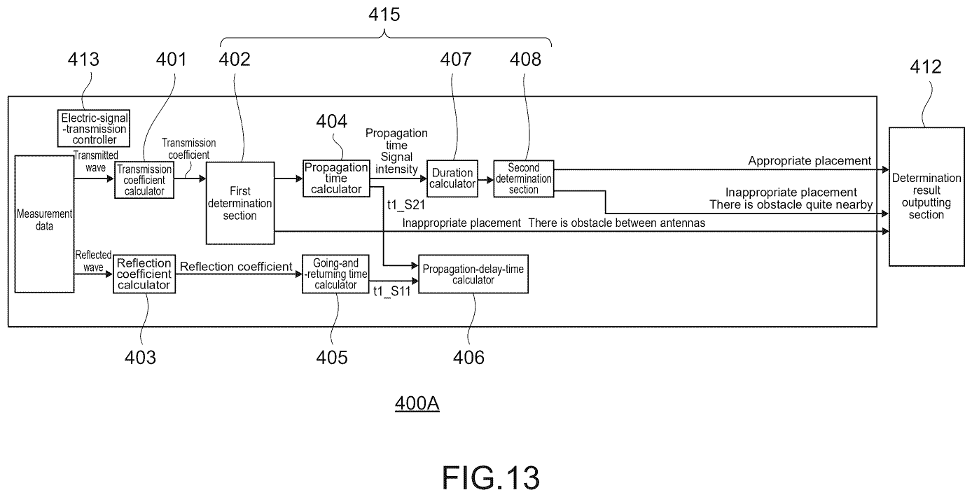

FIG. 13 illustrates a functional configuration of an information processing apparatus according to a first modification.

An information processing apparatus 400A according to the first modification has a configuration obtained by removing the unnecessary wave calculator 414, the third determination section 410, and the position calculator 411 from the information processing apparatus 400 according to the first embodiment. This results in a lower degree of determination probability but in being able to reduce a calculation amount, compared to the case of the first embodiment.

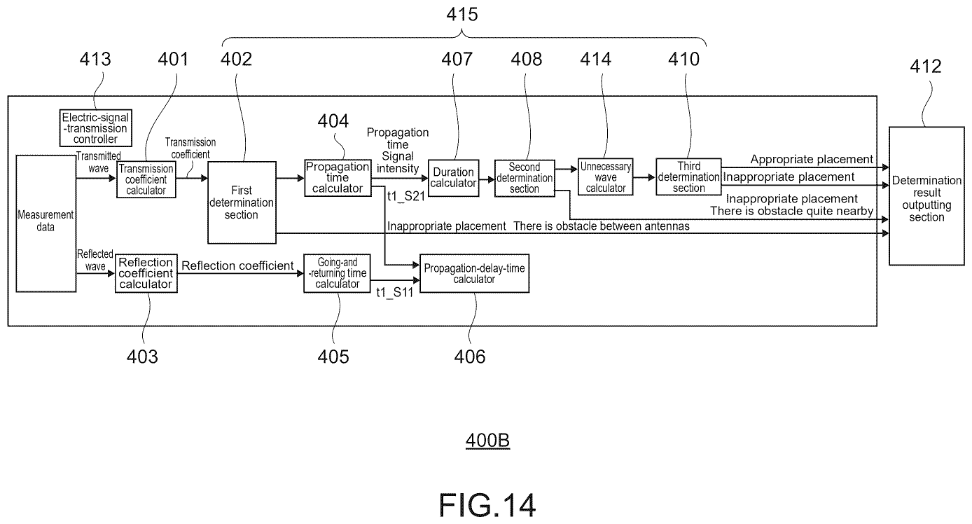

FIG. 14 illustrates a functional configuration of an information processing apparatus according to a second modification.

An information processing apparatus 400B according to the second modification has a configuration obtained by removing the position calculator 411 from the information processing apparatus 400 according to the first embodiment. This results in a lower degree of determination probability but in being able to reduce a calculation amount, compared to the case of the first embodiment.

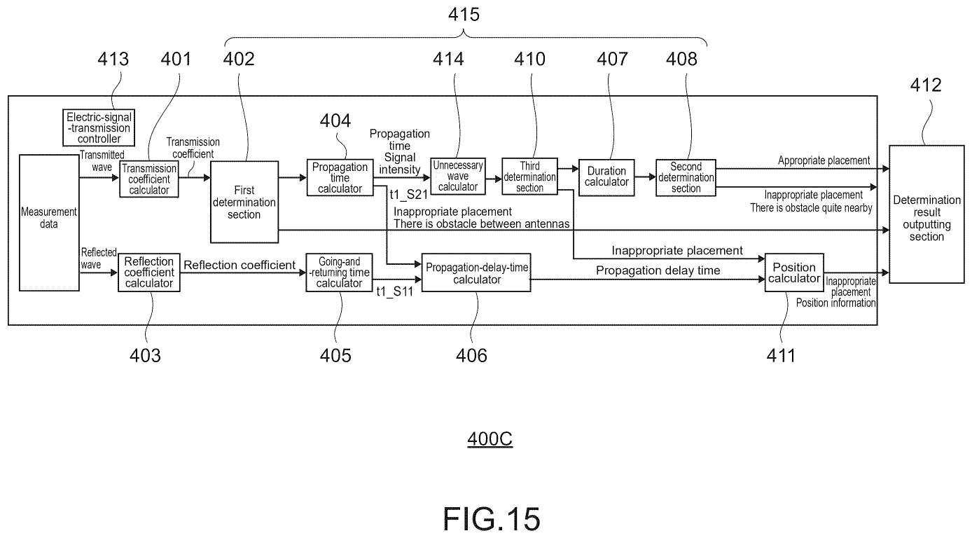

FIG. 15 illustrates a functional configuration of an information processing apparatus according to a third modification.

An information processing apparatus 400C according to the third modification has a configuration in which there is a change in processing order between processing performed by the duration calculator 407 and the second determination section 408 and processing performed by the unnecessary wave calculator 414 and the third determination section 410, compared to the case of the information processing apparatus 400 according to the first embodiment. This results in a degree of determination probability and a calculation amount that are similar to those in the first embodiment.

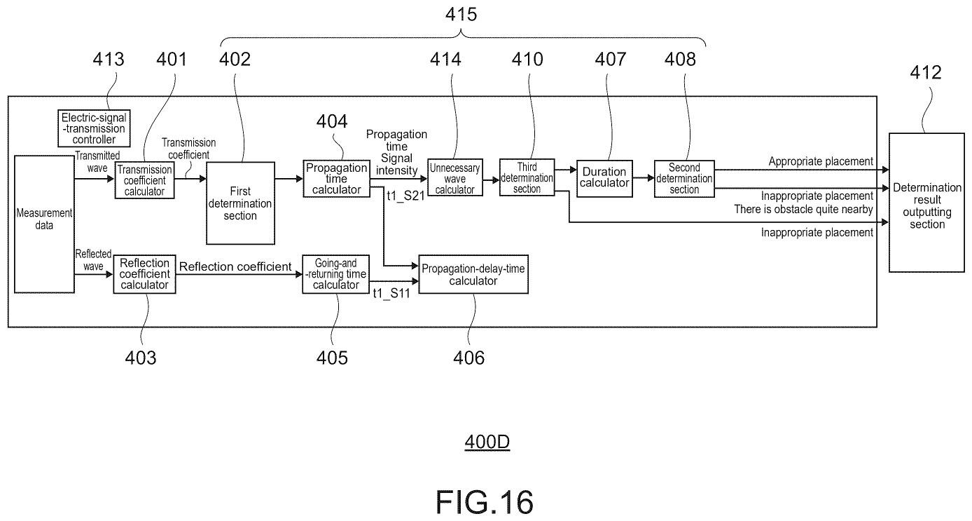

FIG. 16 illustrates a functional configuration of an information processing apparatus according to a fourth modification.

An information processing apparatus 400D according to the fourth modification has a configuration obtained by removing the position calculator 411 from the information processing apparatus 400C according to the third modification. This results in a lower degree of determination probability but in being able to reduce a calculation amount, compared to the case of the third modification.

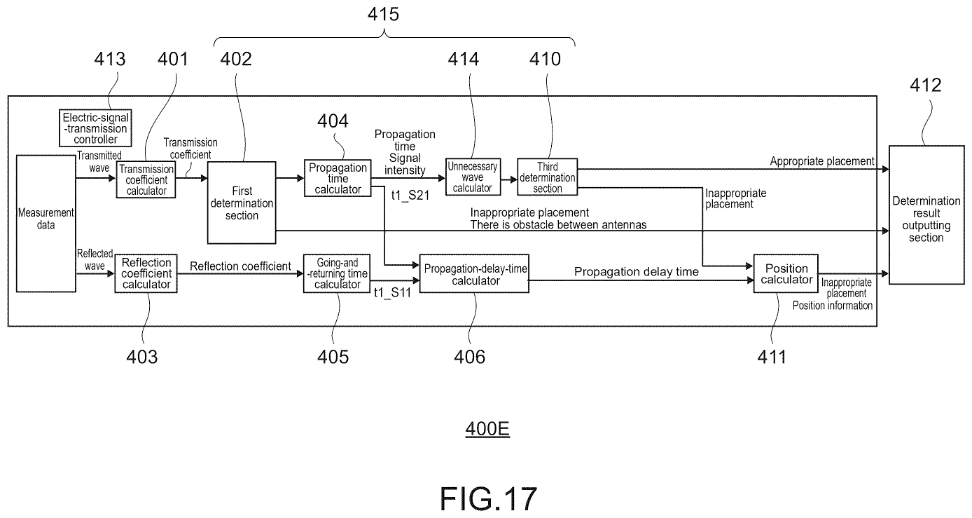

FIG. 17 illustrates a functional configuration of an information processing apparatus according to a fifth modification.

An information processing apparatus 400E according to the fifth modification has a configuration obtained by removing the duration calculator 407 and the second determination section 408 from the information processing apparatus 400 according to the first embodiment. This results in a lower degree of determination probability but in being able to reduce a calculation amount, compared to the case of the first embodiment.

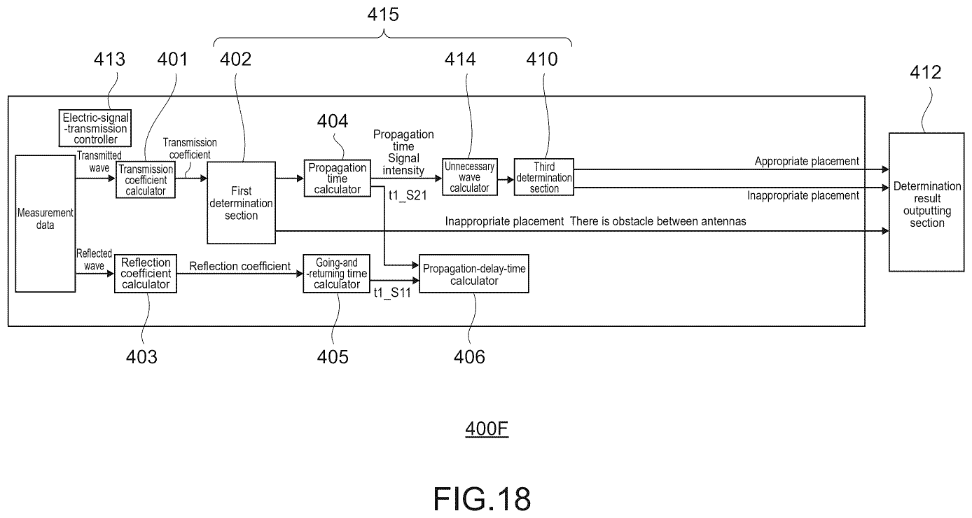

FIG. 18 illustrates a functional configuration of an information processing apparatus according to a sixth modification.

An information processing apparatus 400F according to the sixth modification has a configuration obtained by removing the position calculator 411 from the information processing apparatus 400E according to the fifth modification. This results in a lower degree of determination probability but in being able to reduce a calculation amount, compared to the case of the fifth modification.

According to a seventh modification (not illustrated), not a pair of probes but two pairs of probes may be provided. In this case, the determination section 415 determines whether an object is situated at a specified position in a medium with respect to each of the two pairs of probes 201 and 202 so that the determination section 415 can specify a position of the object on a line but not in a wide range. Further, not a pair of probes but three pairs of probes may be provided. In this case, the determination section 415 determines whether an object is situated at a specified position in a medium with respect to each of the three pairs of probes 201 and 202 so that the determination section 415 can specify a position of an object at a point but not in a wide range. Note that four or more pairs of probes may be provided.

The display apparatus 500 may be placed in the sensor apparatus 110. For example, a placement result may be displayed and represented using an LED, a display, sound, and/or oscillation. There may be a change in, for example, a color of an LED according to the placement result. The determination result outputting section 412 may transmit, by wire, a determination result to another display apparatus 500 that is a device other than the display apparatus 500.

A result of determination performed by the determination section 415 may be stored in, for example, a memory in order to store and use the determination result. The determination result may be transmitted to a signal processing apparatus such as a microcomputer. The determination result may be used to correct a desired peak. The determination result may be used as reliability information for placement. The determination results obtained by determining whether placement is appropriate multiple times, and position measurement performed by, for example, GPS may be combined to display, on a map, good and bad positions for placing a sensor.

The unnecessary wave calculator 414 may correct for an impact of an unnecessary wave. The shape of a function of an unnecessary wave is known. Thus, an impact that the unnecessary wave has on a desired wave can also be removed. For example, the determination section 415 may determine whether placement is appropriate after an impact of an unnecessary wave is removed.

The first embodiment can also be used not only to determine the presence of an obstacle in soil but also to positively detect a useful object in soil.

### 6. Summary

A soil moisture sensor is an apparatus that measures a moisture amount in a medium such as soil, and is used in the fields of agriculture and of soil environment survey. For example, in the field of agriculture, the use of a soil moisture sensor makes it possible to optimally spray crops with water. The soil moisture sensor is used to enhance added values of products.

When an obstacle (an object) is situated around an antenna of a placed soil moisture sensor, there may be a reduction in measurement accuracy. Existing sensors measure propagation delay times of radio waves between antennas. When an obstacle (an object) is situated around the antenna, an unnecessary propagation path may be created, and this may result in inducing an error in propagation delay time. However, it is difficult for a user to determine whether the sensor is placed properly.

On the other hand, according to the first embodiment, the determination section 415 determines an impact that an obstacle has on a measurement result. The position calculator 411 calculates a position of an obstacle (an object). The determination result outputting section 412 displays, on the display apparatus 500, a result of determining whether placement is appropriate.

This enables a user to know whether there is an impact that an obstacle (an object) has on a measurement result. When a position of an obstacle is displayed on the display apparatus 500, this enables a user to estimate an obstacle (an object) and to exclude the displayed position from a placement position. A sensor can be placed after an impact of multipath (two paths that are a desired wave and an unnecessary wave) that occurs due to an obstacle is reduced. This makes it possible to ensure the measurement accuracy.

### II. Second Embodiment

### 1. Overview

In the first embodiment, the information processing apparatus 400 determines whether an object is situated in a specified range in a medium, determines whether placement is appropriate, and presents a result of the determination to a user. On the other hand, in a second embodiment, a moisture distribution in a medium such as soil particularly in a direction of depth in the medium is measured, and the measured moisture distribution is presented to a user. Hereinafter, illustrations and descriptions of a component and an operation that are similar to the component and operation described in the above embodiment and modifications are omitted, and the illustration and the description are given focused on a point different from that described in the above embodiment and modifications.

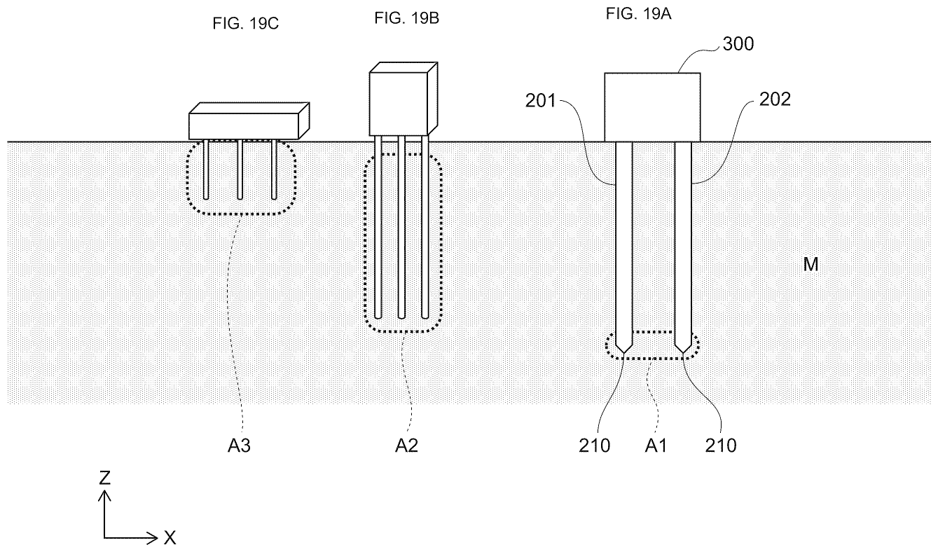

FIG. 19 schematically illustrates the measurement apparatus according to the present embodiment, and comparative examples.

In each of the comparative examples in which antennas are widely provided in an axial direction Z of axes of probes, as illustrated in (B) and (C), a moisture-amount measurement region A2,A3 in the axial direction Z is large, and the respective antennas have a low spatial resolution in the axial direction Z. On the other hand, the respective antennas 210 of the probes 201 and 202 are respectively provided at the respective second ends 204 of the probes 201 and 202, each of the respective second ends 204 being a tip to be inserted into the medium M (FIG. 2). In other words, the respective antennas 210 are not widely provided in the axial direction Z of the probes 201 and 202, but are provided at a precise position in the axial direction Z. Consequently, a region A1 in which the respective antennas 210 measure a moisture amount in the axial direction Z is small, and the respective antennas 210 have a high spatial resolution in the axial direction Z. In other words, the respective antennas 210 according to the present embodiment can measure a moisture amount in a precise region in the axial direction Z, not a moisture amount in the entirety of the medium M in the axial direction Z.

Thus, in the second embodiment, a moisture amount in the medium M is continuously measured for an insertion-and-removal period of time for which a depth of the respective antennas 210 in the medium M keeps on being dynamically changed while the probes 201 and 202 according to the present embodiment are being inserted into and removed from the medium M. This makes it possible to continuously measure moisture amounts at different positions in the medium M in a direction of depth in the medium M when the pair of the respective antennas 210 is situated at the different positions in the depth direction. Consequently, moisture amounts continuously measured at different positions in a direction of depth in the medium M can be presented to a user as a moisture distribution in the depth direction. In particular, the respective antennas 210 are respectively provided to the second ends 204 respectively corresponding to the insertion tips of the probes 201 and 202, and this makes it possible to measure a moisture amount in the medium M over a wider range in the axial direction Z when the probes 201 and 202 are inserted into and removed from the medium M. Note that, in the present embodiment, the “insertion and removal” refers to a movement of keeping on inserting the probes 201 and 202 into the medium M or a movement of pulling the probes 201 and 202 embedded in the medium M out of the medium M.

### 2. Functional Configuration of Information Processing Apparatus

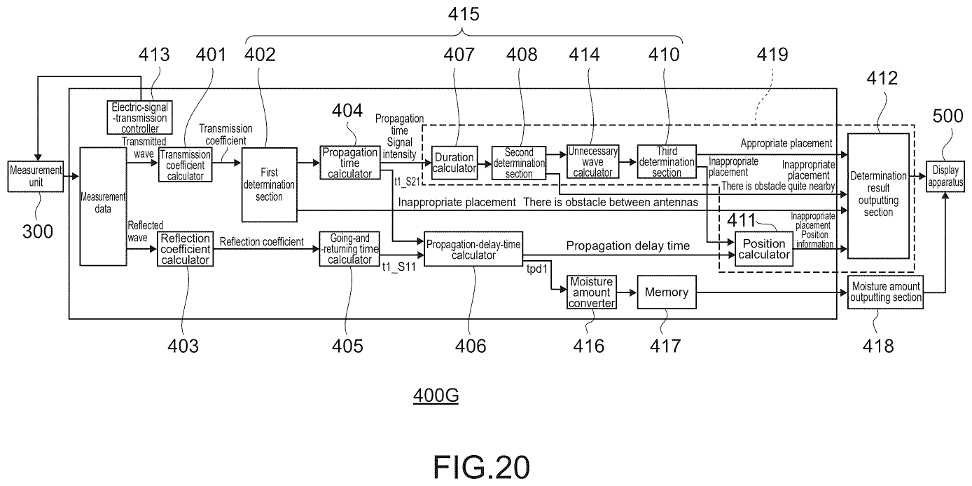

FIG. 20 illustrates a functional configuration of an information processing apparatus according to the second embodiment.

In addition to the functional configuration (FIG. 3) of the information processing apparatus 400 according to the first embodiment, an information processing apparatus 400G according to the second embodiment includes a moisture amount converter 416, a memory 417, and a moisture amount outputting section 418. Note that the information processing apparatus 400G according to the second embodiment does not use the duration calculator 407, the second determination section 408, the unnecessary wave calculator 414, the third determination section 410, the position calculator 411, or the determination result outputting section 412. Thus, the information processing apparatus 400G according to the second embodiment does not necessarily have to include a functional section 419 implemented by these structural elements.

### 3. Flow of Operation of Information Processing Apparatus

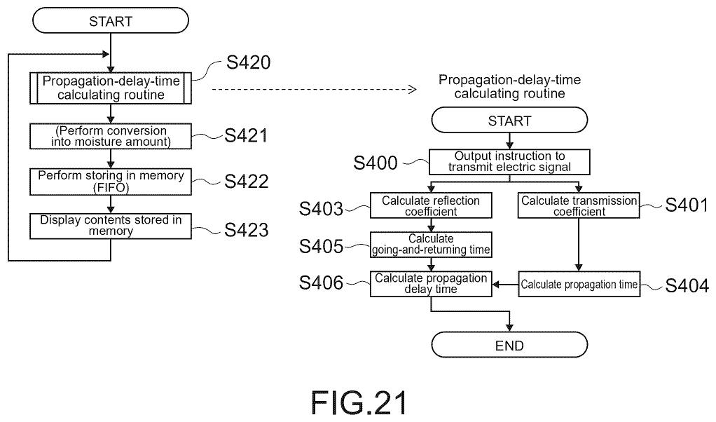

FIG. 21 illustrates a flow of an operation of the information processing apparatus.

It is assumed that a user inserts the pair of probes 201 and 202 into the medium M in the axial direction Z, starting from the side of the respective antennas 210 provided to the respective second ends 204. Consequently, the depth of the respective antennas 210 in the medium M in the axial direction Z keeps on being dynamically changed. The period of time for which the depth of the respective antennas 210 in the medium M in the axial direction Z keeps on being dynamically changed by the probes 201 and 202 being inserted into and removed from the medium M, is referred to as the “insertion-and-removal period of time”.

The information processing apparatus 400G performs a process of a propagation-delay-time calculating routine (Step S420). An operation of the process of the propagation-delay-time calculating routine is similar to the operation performed in the first embodiment (Steps S400, S401, and S403 to S406). Specifically, when a specified trigger occurs (for example, for each specified period of time), the electric-signal-transmission controller 413 outputs, to the transmitter 320 of the measurement unit 300, an instruction that is used to cause the transmitter 320 to transmit an electric signal including an incident wave (Step S400). The transmission coefficient calculator 401 calculates a transmission coefficient from a time waveform of a transmitted wave received by the transmitted wave receiver 350 (Step S401). The reflection coefficient calculator 403 calculates a reflection coefficient from a time waveform of a reflected wave received by the reflected wave receiver 340 (a reflected wave obtained by an incident wave being reflected off the probe 201) (Step S403) The propagation time calculator 404 calculates a propagation time and a signal intensity of a transmitted wave, and calculates a signal intensity A and a time t_{1m_S21} at a peak of a desired wave (Step S404). The going-and-returning time calculator 405 calculates a going-and-returning time and a signal intensity of a reflected wave, and calculates a signal intensity and a time t_{1m_S11} at a peak of the reflected wave (Step S405). The propagation-delay-time calculator 406 calculates a propagation delay time t_{pd1} that is a difference between the time t_{1m_S21} at the peak of a desired wave and the time t_{1m_S11} at the peak of a reflected wave, the propagation delay time t_{pd1} being obtained by subtracting the time t_{1m_S11} from the time t_{1m_S21} (Step S406). The propagation delay time is a time for which an electromagnetic wave propagates through a region, in the medium, that is situated between the probes 201 and 202.

The moisture amount converter 416 converts the propagation delay time t_{pd1} (Step S406) into a moisture amount in a medium using a coefficient that represents a relationship between the moisture amount and the propagation delay time t_{pd1} (Step S421). The moisture amount converter 416 continuously obtains, for each specified time, a moisture amount in the medium M for the insertion-and-removal period of time. Using the first-in, first-out method (FIFO), the moisture amount converter 416 chronologically records, in the memory 417, the moisture amounts obtained for the insertion-and-removal period of time (Step S422).

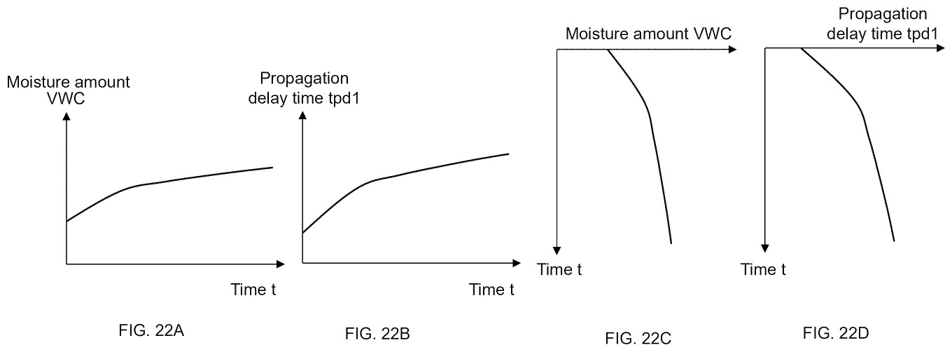

FIG. 22 illustrates examples of display performed by the moisture amount outputting section.

In the form of, for example, a graph, the moisture amount outputting section 418 chronologically displays, on the display apparatus 500, a history of the moisture amounts obtained for the insertion-and-removal period of time and chronologically recorded in the memory 417 (Step S423). Specifically, as illustrated in (A), the moisture amount outputting section 418 displays, on the display apparatus 500, a graph having two axes that respectively represent a history of moisture amounts (volume water content) chronologically recorded in the memory 417 and an elapsed time corresponding to the chronological order for the recording. Note that the moisture amounts are chronologically recorded in the memory 417 using the first-in, first-out method (FIFO). Thus, only a history of most recent moisture amounts stored in the memory 417 is displayed on the graph. Accordingly, a moisture amount that is contained in a medium and corresponds to a depth of the respective antennas 210 that is substantially proportional to an elapsed time for inserting and removing the probes 201 and 202, can be displayed in an intuitive manner for a user.

As a modification, the moisture amount outputting section 418 may display, on the display apparatus 500, a graph having two axes that respectively represent the history of the moisture amounts chronologically recorded in the memory 417 and the chronologically changed depth instead of the elapsed time corresponding to the chronological order for the recording. For example, it is sufficient if the moisture amount outputting section 418 estimates depth positions of the pair of the respective antennas 210 at respective time points at which the elapsed time is measured, the depth position being obtained by the pair of the respective antennas 210 displacing chronologically, the estimation being performed on the basis of an entire length of the probe 201,202 (that is, a maximum depth of insertion) and on the basis of an elapsed time from a starting time point to a finishing time point of the insertion-and-removal period of time. In other words, it is sufficient if, substantially in proportion to the time elapsed since the start of the insertion-and-removal period of time, the moisture amount outputting section 418 estimates the depth positions obtained by the chronological displacement being performed for the insertion-and-removal period of time.

As a modification, instead of the history of moisture amounts, the moisture amount outputting section 418 may display, on a graph, a history of propagation delay times respectively corresponding to the moisture amounts, as illustrated in (B). The moisture amount is calculated from the propagation delay time t_{pd1} using a coefficient. Thus, a line graph of the propagation delay time t_{pd1} has characteristics similar to characteristics of a line graph of the moisture amount. Thus, display of a propagation delay time on a graph instead of a moisture amount also enables a user to understand characteristics of a moisture distribution in the depth direction. Note that, when the history of propagation delay times is displayed on a graph instead of the history of moisture amounts, the process of Step S421 may be omitted.

Further, the vertical axis and the horizontal axis of the graph illustrated in (A) may be reversed to obtain a graph having a horizontal axis that represents the history of moisture amounts and a vertical axis that represents the elapsed time, and the obtained graph may be displayed on the display apparatus 500, as illustrated in (C). Furthermore, the vertical axis and the horizontal axis of the graph illustrated in (B) may be reversed to obtain a graph having a horizontal axis that represents the history of propagation delay times tₚdl and a vertical axis that represents the elapsed time, and the obtained graph may be displayed on the display apparatus 500, as illustrated in (D). When the elapsed time is represented by the vertical axis, as in the case of these graphs, a depth of the respective antennas 210 that is substantially proportional to the elapsed time can be displayed in an intuitive manner for a user.

### 4. Examples

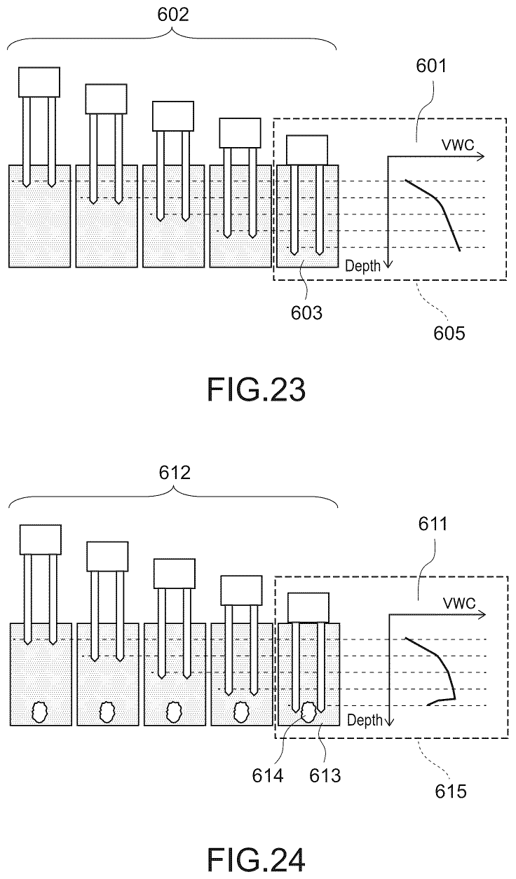

FIG. 23 schematically illustrates a graph of a moisture amount and a relationship of displacement of a depth of antennas to the graph of the moisture amount according to a first example.

In this example, the moisture amount outputting section 418 displays, on the display apparatus 500, a graph 601 having two axes that respectively represent a history of moisture amounts chronologically recorded in the memory 417 and a depth position obtained by chronological displacement being performed for the insertion-and-removal period of time. In the figure, a moisture amount in the medium M at a depth position at which the respective antennas 210 respectively provided to the insertion tips of the respective probes 201 and 202 are situated during insertion of the probes 201 and 202 into the medium M, is precisely displayed in the form of a graph. The moisture amount outputting section 418 may further cause a GUI 602 and a GUI 603 to be displayed in addition to the display of the graph 601, where the GUI 602 is a chronological GUI that shows the process of inserting the probes 201 and 202 into the medium M, and the GUI 603 shows a state in which the probes 201 and 202 have been completely inserted into the medium M. Here, the moisture amount outputting section 418 favorably causes a GUI 605 to be displayed, where the vertical axis (the depth) of the graph 601 and an insertion depth to which the respective antennas 210 are inserted on the GUI 602 or 603, are arranged side by side on the GUI 605 such that the vertical axis of the graph 601 and the insertion depth can be visually associated with each other. This enables a user to intuitively understand a depth position of the respective antennas 210 in the medium M that is obtained for the insertion-and-removal period of time, and a moisture amount that corresponds to the depth position of the respective antennas 210. Note that the graph 601 of the first example exhibits a moisture distribution showing that the moisture amount is larger at a deeper position in the medium M.

FIG. 24 schematically illustrates a graph of a moisture amount and a relationship of displacement of a depth of antennas to the graph of the moisture amount according to a second example.

A graph 611 of the second example exhibits a moisture distribution showing that the moisture amount is larger at a deeper position in the medium M, as in the case of the first example. On the other hand, there is a great reduction in moisture amount around a depth position at which the respective antennas 210 are situated when the probes 201 and 202 have been completely inserted into the medium M (a GUI 613). This means that an obstacle (an object) such as a stone may be situated around the depth position at which the respective antennas 210 are situated when the probes 201 and 202 have been completely inserted into the medium M (the GUI 613). The moisture amount outputting section 418 may further cause a GUI 612 and the GUI 613 to be displayed in addition to the display of the graph 611, where the GUI 612 is a chronological GUI that shows the process of inserting the probes 201 and 202 into the medium M, and the GUI 613 shows a state in which the probes 201 and 202 have been completely inserted into the medium M. Here, the moisture amount outputting section 418 may display, on the display apparatus 500, information that indicates a depth of an object situated in the medium M, the depth of the object being estimated on the basis of a change in moisture amount relative to the depth of the respective antennas 210 in the medium M. Specifically, here, the moisture amount outputting section 418 favorably causes a GUI 615 to be displayed, where the vertical axis (the depth) of the graph 611, an insertion depth to which the respective antennas 210 are inserted on the GUI 612 or 613, and a depth position at which an object 614 may be situated, are arranged side by side on the GUI 615 such that the vertical axis of the graph 611, the insertion depth, and the depth position can be visually associated with each other. This enables a user to intuitively understand a depth position of the respective antennas 210 in the medium M that is obtained for the insertion-and-removal period of time, a depth position at which the object 614 may be situated, and a moisture amount that corresponds to the depth position of the respective antennas 210. Further, the user can take measures such as inserting the probes 201 and 202 at another position or placing the probes 201 and 202 in a state of slightly protruding from the medium M without being completely inserted, such that the probes 201 and 202 are not affected by the object 614.

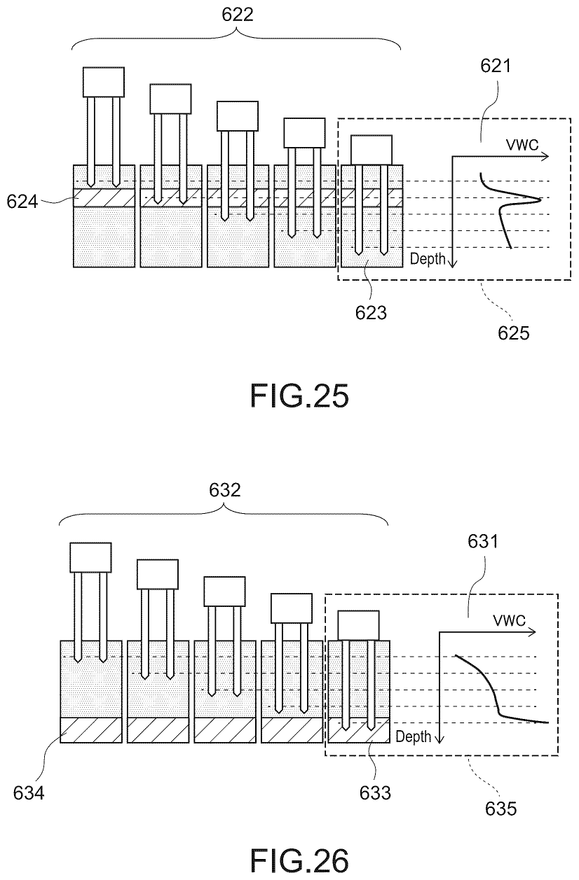

FIG. 25 schematically illustrates a graph of a moisture amount and a relationship of displacement of a depth of antennas to the graph of the moisture amount according to a third example.

A graph 621 of the third example exhibits a moisture distribution showing that the moisture amount is larger at a deeper position in the medium M, as in the case of the first example. On the other hand, there is a great increase in moisture amount around a depth position at which the respective antennas 210 are situated when the probes 201 and 202 have been inserted into the medium M to a depth corresponding to about ⅖ of a length of the probe 201,202. This means that a heterogeneous medium 624 that contains a significantly large moisture amount may be situated around the depth position at which the respective antennas 210 are situated when the probes 201 and 202 have been inserted into the medium M to the depth corresponding to about ⅖ of the length of the probe 201,202. The moisture amount outputting section 418 may further cause a GUI 622 and a GUI 623 to be displayed in addition to the display of the graph 621, where the GUI 622 is a chronological GUI that shows the process of inserting the probes 201 and 202 into the medium M, and the GUI 623 shows a state in which the probes 201 and 202 have been completely inserted into the medium M. Here, the moisture amount outputting section 418 favorably causes a GUI 625 to be displayed, where the vertical axis (the depth) of the graph 621, an insertion depth to which the respective antennas 210 are inserted on the GUI 622 or 623, and a depth position at which the heterogeneous object 624 may be situated, are arranged side by side on the GUI 625 such that the vertical axis of the graph 621, the insertion depth, and the depth position can be visually associated with each other. This enables a user to intuitively understand a depth position of the respective antennas 210 in the medium M that is obtained for the insertion-and-removal period of time, a depth position at which the heterogeneous object 624 may be situated, and a moisture amount that corresponds to the depth position of the respective antennas 210. Further, the user can take measures such as inserting the probes 201 and 202 at another position or placing the probes 201 and 202 in a state of being inserted into the medium M to a depth corresponding to about ⅕ of the length of the probe 201,202 without being completely inserted, such that the probes 201 and 202 are not affected by the heterogeneous object 624.

FIG. 26 schematically illustrates a graph of a moisture amount and a relationship of displacement of a depth of antennas to the graph of the moisture amount according to a fourth example.

A graph 631 of the fourth example exhibits a moisture distribution showing that the moisture amount is larger at a deeper position in the medium M, as in the case of the first example. On the other hand, there is a great increase in moisture amount around a depth position at which the respective antennas 210 are situated when the probes 201 and 202 have been completely inserted into the medium M (a GUI 633). This means that a heterogeneous medium 634 that contains a significantly large moisture amount may be situated around the depth position at which the respective antennas 210 are situated when the probes 201 and 202 have been completely inserted into the medium M (the GUI 633). The moisture amount outputting section 418 may further cause a GUI 632 and the GUI 633 to be displayed in addition to the display of the graph 631, where the GUI 632 is a chronological GUI that shows the process of inserting the probes 201 and 202 into the medium M, and the GUI 633 shows a state in which the probes 201 and 202 have been completely inserted into the medium M. Here, the moisture amount outputting section 418 favorably causes a GUI 635 to be displayed, where the vertical axis (the depth) of the graph 631, an insertion depth to which the respective antennas 210 are inserted on the GUI 632 or 633, and a depth position at which the heterogeneous medium 634 may be situated, are arranged side by side on the GUI 635 such that the vertical axis of the graph 631, the insertion depth, and the depth position can be visually associated with each other. This enables a user to intuitively understand a depth position of the respective antennas 210 in the medium M that is obtained for the insertion-and-removal period of time, a depth position at which the heterogeneous medium 634 may be situated, and a moisture amount that corresponds to the depth position of the respective antennas 210. Further, the user can take measures such as inserting the probes 201 and 202 at another position or placing the probes 201 and 202 in a state of slightly protruding from the medium M without being completely inserted, such that the probes 201 and 202 are not affected by the heterogeneous medium 634.

### 5. Summary

The respective antennas 210 are not widely provided in the axial direction Z of the probes 201 and 202, but are provided at a precise position in the axial direction Z. Consequently, a region in which the respective antennas 210 according to the present embodiment measure a moisture amount in the axial direction Z is smaller, and the respective antennas 210 have a higher spatial resolution in the axial direction Z, compared to when antennas are provided widely in the axial direction Z of axes of probes. In other words, the respective antennas 21 according to the present embodiment can measure a moisture amount in a precise region in the axial direction Z, not a moisture amount in the entirety of a medium in the axial direction Z. Thus, in the second embodiment, a moisture amount in the medium is continuously measured for the insertion-and-removal period of time for which a depth of the respective antennas 210 in the medium M keeps on being dynamically changed while the probes 201 and 202 according to the present embodiment are being inserted into and removed from the medium. This makes it possible to continuously measure moisture amounts at different positions in a medium in a direction of depth in the medium when the pair of the respective antennas 210 is situated at the different positions in the depth direction. Consequently, moisture amounts continuously measured at different positions in a direction of depth in a medium can be presented to a user as a moisture distribution in the depth direction.

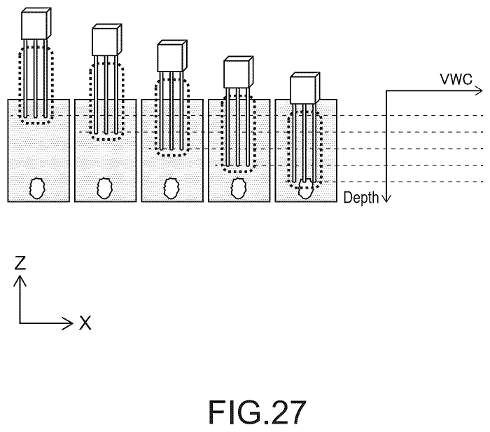

FIG. 27 illustrates a moisture sensor according to a comparative example.

On the other hand, antennas are widely provided in an axial direction Z of axes of probes in the case of typical moisture sensors. Thus, the antennas have a low spatial resolution since measurement is performed on a region around the rod-shaped probes at a time. For this reason, measurement is performed averagely and thus is relatively less affected by an object such as an extraneous material. In other words, it is difficult to detect an object such as an extraneous material. Further, it is difficult to accurately acquire a moisture distribution in the depth direction due to the low spatial resolution in the axial direction Z. In the case in which antennas are widely provided in an axial direction Z of axes of probes, a measurement range in the axial direction Z is large. This results in difficulty in measuring moisture amounts at different positions in the depth direction even if a moisture amount that is contained in a medium and obtained for the insertion-and-removal period of time is continuously measured. Thus, it is difficult to measure a moisture distribution in a direction of depth in the medium. Therefore, with respect to sensors in which antennas are widely provided in an axial direction Z of axes of probes, it is less necessary that moisture be measured fast and display be performed chronologically in the process of inserting the sensor in a medium.

### 6. Modifications

### 6-1. Eighth Modification

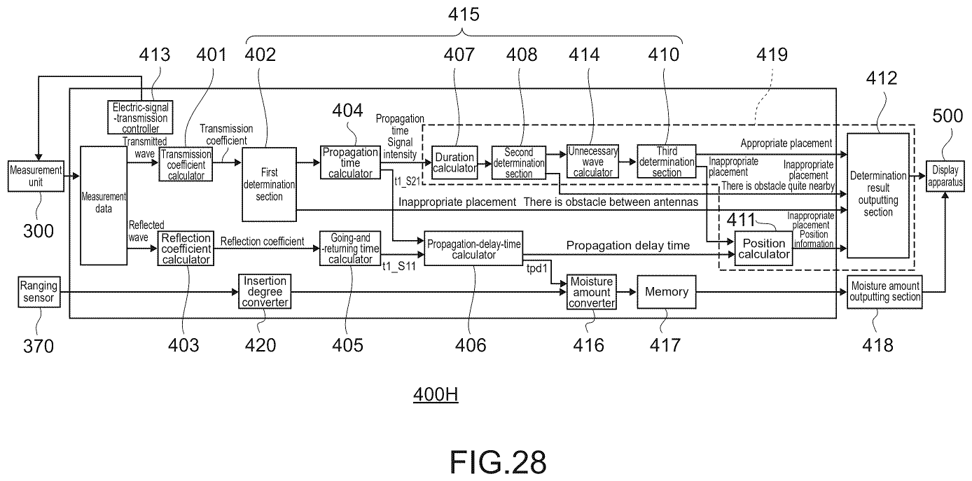

FIG. 28 illustrates a functional configuration of an information processing apparatus according to an eighth modification.

An information processing apparatus 400H according to the eighth modification has a configuration obtained by adding an insertion degree converter 420 to the information processing apparatus 400G according to the second embodiment. Further, a ranging sensor 370 is provided to the measurement unit 300.

The ranging sensor 370 measures a distance to the surface of a medium from the measurement unit 300. It is sufficient if the ranging sensor 370 is a contactless sensor such as a laser positioning calculator, a ToF sensor, a stereo camera, an ultrasonic sensor, or a radar; or a mechanical sensor such as a contact displacement gauge. The use of the ranging sensor 370 makes it possible to obtain a depth in a medium more accurately, compared to the case of the second embodiment.

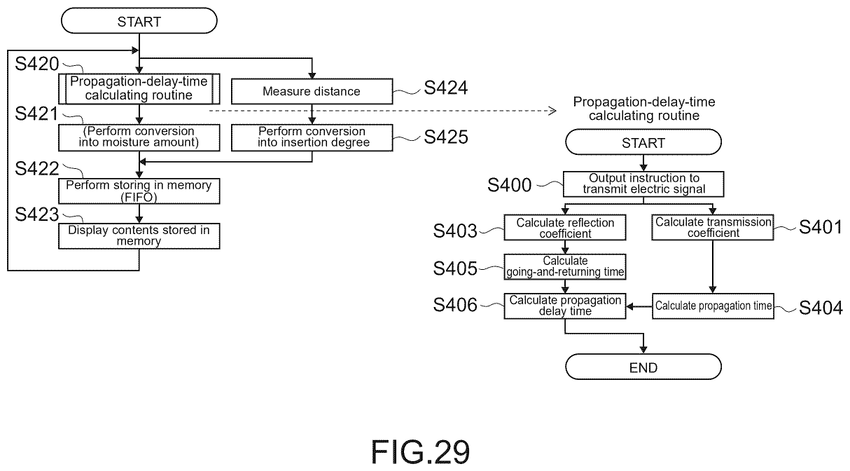

FIG. 29 illustrates a flow of an operation of the information processing apparatus.

It is assumed that a user inserts the pair of probes 201 and 202 into the medium M in the axial direction Z, starting from the side of the respective antennas 210 provided to the respective second ends 204. Consequently, the depth of the respective antennas 210 in the medium M in the axial direction Z keeps on being dynamically changed. In other words, a distance to the surface of the medium M from the measurement unit 300 keeps on being dynamically changed. The ranging sensor 370 measures the distance to the surface of the medium M from the measurement unit 300 (Step S424).

The insertion degree converter 420 converts, into a depth of the respective antennas 210 in the medium M (a degree of insertion of the pair of probes 201 and 202), the distance measured by the ranging sensor 370 and dynamically changed according to the degree of insertion of the pair of probes 201 and 202 (Step S425).

On the other hand, the information processing apparatus 400H performs the process of the propagation-delay-time calculating routine (Step S420). An operation of the process of the propagation-delay-time calculating routine is similar to the operation performed in the second embodiment (Steps S400, S401, and S403 to S406). The moisture amount converter 416 converts the propagation delay time t_{pd1} (Step S406) into a moisture amount in a medium using a coefficient that represents a relationship between the moisture amount and the propagation delay time t_{pd1} (Step S421). The moisture amount converter 416 continuously obtains, for each specified time, a moisture amount in the medium M for the insertion-and-removal period of time. Using the first-in, first-out method (FIFO), the moisture amount converter 416 chronologically records, in the memory 417 and in association with the depths (Step S425) of the pair of the respective antennas 210 in the medium M, the moisture amounts obtained for the insertion-and-removal period of time (Step S422).

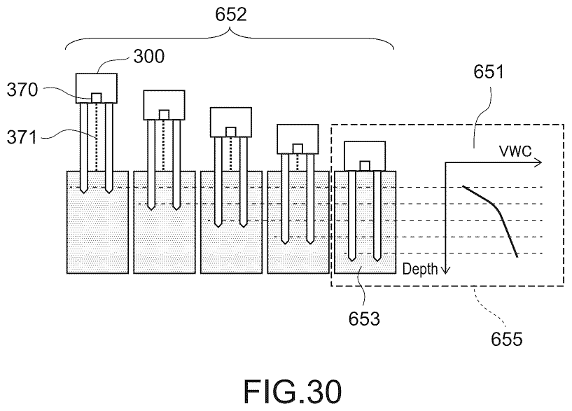

FIG. 30 schematically illustrates a graph of a moisture amount and a relationship of displacement of a depth of antennas to the graph of the moisture amount.

In the form of, for example, a graph, the moisture amount outputting section 418 chronologically displays, on the display apparatus 500, a history of the moisture amounts obtained for the insertion-and-removal period of time and chronologically recorded in the memory 417 (Step S423). Specifically, the moisture amount outputting section 418 displays, on the display apparatus 500, a graph 651 having two axes that respectively represent a history of moisture amounts (volume water content) chronologically recorded in the memory 417 and the chronologically changed depth. In the figure, a moisture amount in the medium M at a depth position at which the respective antennas 210 respectively provided to the insertion tips of the respective probes 201 and 202 are situated during insertion of the probes 201 and 202 into the medium M, is precisely displayed in the form of a graph. The moisture amount outputting section 418 may further cause a GUI 652 and a GUI 653 to be displayed in addition to the display of the graph 651, where the GUI 652 is a chronological GUI that shows the process of inserting the probes 201 and 202 into the medium M, and the GUI 653 shows a state in which the probes 201 and 202 have been completely inserted into the medium M. The chronological GUI 652 showing the process of inserting the probes 201 and 202 into the medium M may further include the ranging sensor 370 and a laser 371 provided when the ranging sensor 370 is a laser positioning calculator. Here, the moisture amount outputting section 418 favorably causes a GUI 655 to be displayed, where the vertical axis (the depth) of the graph 651 and an insertion depth to which the respective antennas 210 are inserted on the GUI 652 or 653, are arranged side by side on the GUI 655 such that the vertical axis of the graph 651 and the insertion depth can be visually associated with each other. This enables a user to intuitively understand a depth position of the respective antennas 210 in the medium M that is obtained for the insertion-and-removal period of time, and a moisture amount that corresponds to the depth position of the respective antennas 210. Note that the graph 651 exhibits a moisture distribution showing that the moisture amount is larger at a deeper position in the medium M.

### 6-2. Ninth Modification

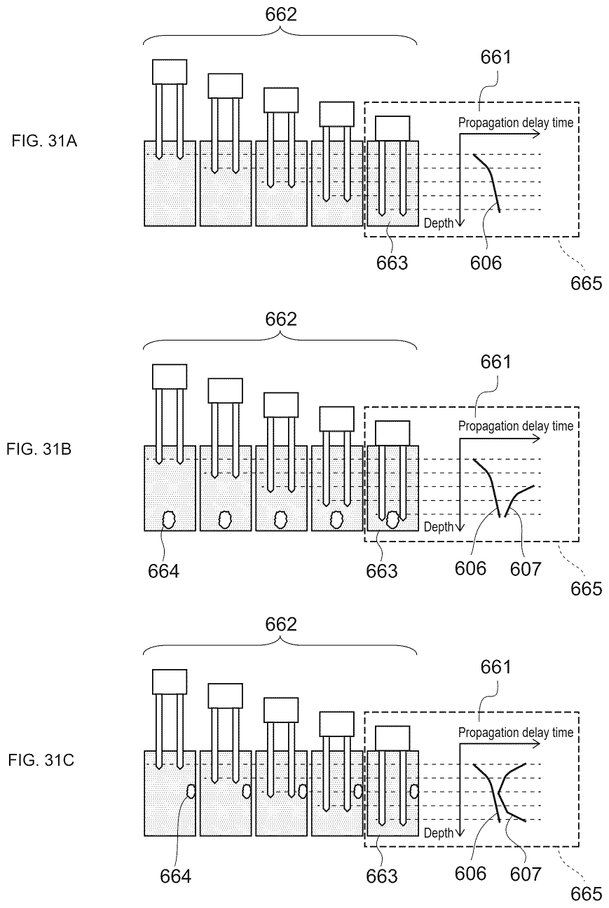

FIG. 31 illustrates a ninth modification.

As described above, instead of the history of moisture amounts, the moisture amount outputting section 418 may display, on a graph, a history of desired-wave propagation delay times respectively corresponding to the moisture amounts, where the desired-wave propagation delay time is a chronologically changed propagation delay time of a desired wave. Here, the moisture amount outputting section 418 may further display a history of propagation delay times of an unnecessary wave on the graph.

The propagation delay time is a difference between the time t_{1m_S21} at a peak of a transmitted wave and the time t_{1m_S11} at a peak of a reflected wave. When no object is situated in a specified range, no unnecessary wave is caused. Thus, only the peak of a desired wave is detected. Thus, when no object is situated in the specified range, only a desired-wave propagation delay time 606 that is a propagation delay time of a desired wave (direct wave) is displayed, as described in (A).

On the other hand, when an object is situated in the specified range, an unnecessary wave is detected. Thus, the peak of a desired wave and the peak of an unnecessary wave are detected. The propagation delay time of an unnecessary wave is different from the desired-wave propagation delay time 606 corresponding to the propagation delay time of a desired wave. In this case, the information processing apparatus 400G or 400H performs the processes of Steps S420 to S423, S404, and S406 for the peaks of a desired wave and an unnecessary wave. The moisture amount outputting section 418 further displays, on the graph, a propagation delay time 607 of an unnecessary wave in addition to the desired-wave propagation delay time 606, as illustrated in (B) and (C). When only the desired-wave propagation delay time 606 is displayed without the propagation delay time 607 being displayed, this enables a user to intuitively understand that the position is a desirable placement position since no object is situated in the specified range. On the other hand, when the propagation delay time 607 is displayed in addition to the desired-wave propagation delay time 606, this enables the user to intuitively understand that it is better to change the placement position since an object is situated in the specified range.

In this modification, the moisture amount outputting section 418 may also further cause a GUI 662 and a GUI 663 to be displayed in addition to the display of a graph 661, as in the case of, for example, FIG. 24, where the GUI 662 is a chronological GUI that shows the process of inserting the probes 201 and 202 into the medium M, and the GUI 663 shows a state in which the probes 201 and 202 have been completely inserted into the medium M. Here, the moisture amount outputting section 418 may display, on the display apparatus 500, information that indicates a depth of an object situated in the medium M, the depth of the object being estimated on the basis of a change in moisture amount relative to the depth of the respective antennas 210 in the medium M. Specifically, here, the moisture amount outputting section 418 favorably causes a GUI 665 to be displayed, where the vertical axis (the depth) of the graph 661, an insertion depth to which the respective antennas 210 are inserted on the GUI 662 or 663, and a depth position at which an object 664 may be situated, are arranged side by side on the GUI 665 such that the vertical axis of the graph 661, the insertion depth, and the depth position can be visually associated with each other. This enables a user to intuitively understand a depth position of the respective antennas 210 in the medium M that is obtained for the insertion-and-removal period of time, a depth position at which the object 664 may be situated, and a moisture amount that corresponds to the depth position of the respective antennas 210. Further, the user can take measures such as inserting the probes 201 and 202 at another position or placing the probes 201 and 202 in a state of slightly protruding from the medium M without being completely inserted, such that the probes 201 and 202 are not affected by the object 664.

### 6-3. Tenth Modification

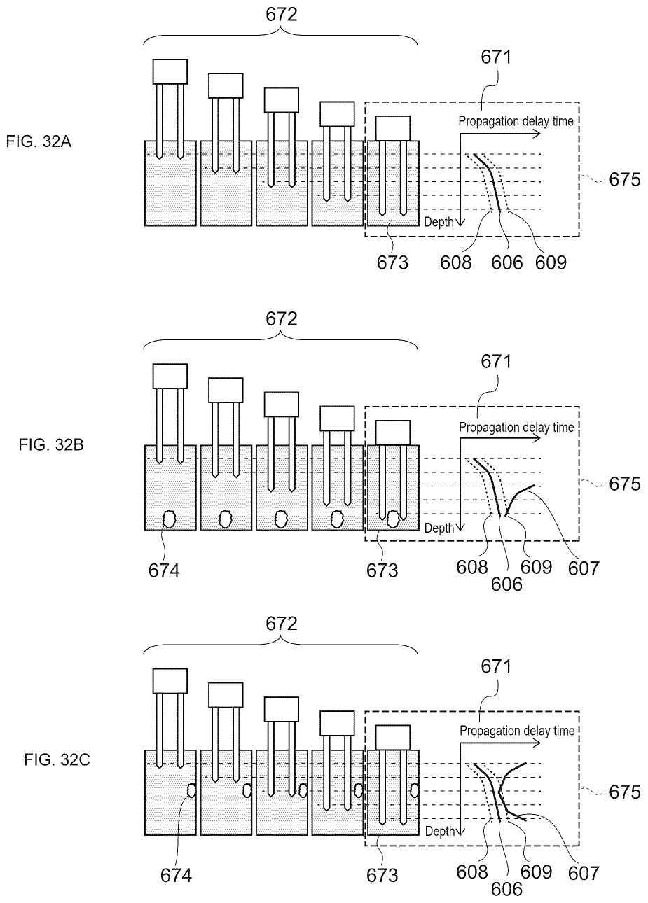

FIG. 32 illustrates a tenth modification.

The tenth modification is obtained by applying the ninth modification. The moisture amount outputting section 418 may further display, on the graph, a range in which calculation of the desired-wave propagation delay time 606 may be affected by an object being situated in a specified range in a medium with respect to the pair of probes 201 and 202, the range being determined using the desired-wave propagation delay time 606 as a reference. Graphs illustrated in (A), (B), and (C) of FIG. 32 are each obtained by lines 608 and 609 being further displayed on a corresponding one of the graphs illustrated in (A), (B), and (C) of FIG. 31 using the desired-wave propagation delay time 606 as a reference, where the lines 608 and 609 indicate a range (an NG region) in which the desired-wave propagation delay time 606 may be affected. Using the desired-wave propagation delay time 606 as a reference, the lines 608 and 609 indicate a range of a value of a propagation delay time, where a value of the desired-wave propagation delay time 606 is in the middle in the range of the value of the propagation delay time. When at least a portion of values of the propagation delay time 607 of an unnecessary wave are in the range (the NG region) formed between the lines 608 and 609, this means that an object may be situated in a specified range in a medium with respect to the pair of probes 201 and 202. When at least a portion of the values of the propagation delay time 607 of an unnecessary wave are in the range (the NG region) formed between the lines 608 and 609, this enables a user to intuitively understand that it is better to change the placement position since an object is situated in the specified range.

In this modification, the moisture amount outputting section 418 may also further cause a GUI 672 and a GUI 673 to be displayed in addition to the display of a graph 671, as in the case of FIG. 24, where the GUI 672 is a chronological GUI that shows the process of inserting the probes 201 and 202 into the medium M, and the GUI 673 shows a state in which the probes 201 and 202 have been completely inserted into the medium M. Here, the moisture amount outputting section 418 may display, on the display apparatus 500, information that indicates a depth of an object situated in the medium M, the depth of the object being estimated on the basis of a change in moisture amount relative to the depth of the respective antennas 210 in the medium M. Specifically, here, the moisture amount outputting section 418 favorably causes a GUI 675 to be displayed, where the vertical axis (the depth) of the graph 671, an insertion depth to which the respective antennas 210 are inserted on the GUI 672 or 673, and a depth position at which an object 674 may be situated, are arranged side by side on the GUI 675 such that the vertical axis of the graph 671, the insertion depth, and the depth position can be visually associated with each other. This enables a user to intuitively understand a depth position of the respective antennas 210 in the medium M that is obtained for the insertion-and-removal period of time, a depth position at which the object 674 may be situated, and a moisture amount that corresponds to the depth position of the respective antennas 210. Further, the user can take measures such as inserting the probes 201 and 202 at another position or placing the probes 201 and 202 in a state of slightly protruding from the medium M without being completely inserted, such that the probes 201 and 202 are not affected by the object 674.

### 6-4. Eleventh Modification

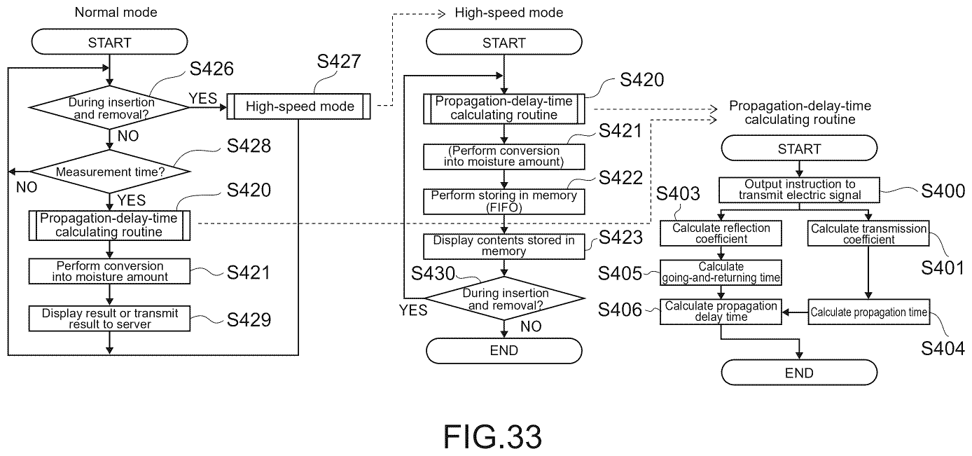

FIG. 33 illustrates a flow of an operation according to an eleventh modification.

The information processing apparatus 400G may measure a moisture amount frequently during insertion and removal of the pair of probes 201 and 202 (a high-speed mode), and may measure the moisture amount less frequently when the pair of probes 201 and 202 is in a non-dynamic state (a normal mode), compared to the case in which the frequent measurement is performed. The normal mode is used for normal measurement performed when the accuracy is emphasized. The normal mode is a mode that is used in a stable state in which there is not a frequent dynamic change in position and used to calculate a moisture amount on the basis of a larger number of measurement results (a large number of measurement results are used to perform averaging). The high-speed mode is used for measurement performed for the insertion-and-removal period of time when the speed is emphasized. The high-speed mode is a mode that is used to check temporary characteristics and used to calculate a moisture amount on the basis of fewer measurement results while sacrificing the accuracy to some extent (a small number of measurement results are used to perform averaging). The information processing apparatus 400G includes a mode switcher (not illustrated). The mode switcher may automatically switch modes on the basis of output from the ranging sensor 370 or output from, for example, an acceleration sensor (not illustrated) that is provided to the measurement unit 300. Alternatively, a user may manually perform an operation for mode switching, and the mode switcher may detect the operation and switch modes. The switching of modes makes it possible to perform optimal measurement according to the dynamic or non-dynamic state of the probes 201 and 202. During insertion and removal, it will be difficult to follow a change unless results are frequently displayed at a high speed. Conversely, there is not a frequent change when the probes 201 and 202 remain placed. Thus, it is sufficient if display is less frequently performed at a low speed. Typically, averaging processing is performed in order to increase the measurement accuracy. However, upon performing a high-speed display, results are displayed frequently by reducing the number of samples used for averaging. Upon performing a low-speed display, results are displayed less frequently by increasing the number of samples used for averaging.

On the basis of, for example, output from the ranging sensor 370, output from, for example, an acceleration sensor, or an operation performed by a user, the information processing apparatus 400G determines whether the pair of probes 201 and 202 is in a state during the insertion-and-removal period of time (Step S426). When it has been determined that the pair of probes 201 and 202 is in the state during the insertion-and-removal period of time (YES in Step S426), the information processing apparatus 400G starts the high-speed mode (Step S427). In the high-speed mode, the information processing apparatus 400G measures a moisture amount frequently, and keeps on chronologically displaying, on the display apparatus 500, a history of the moisture amounts obtained for the insertion-and-removal period of time, the display being performed in the form of, for example, a graph (Steps S420 to S423). Until the mode switcher determines that the pair of probes 201 and 202 is not in the state during the insertion-and-removal period of time but in a stable state of being placed (NO in Step S430), the information processing apparatus 400G measures a moisture amount frequency, and keeps on causing the history of the moisture amounts obtained for the insertion-and-removal period of time to be chronologically displayed.

On the other hand, when the pair of probes 201 and 202 is not in the state during the insertion-and-removal period of time but in the stable state of being placed (NO in Step S430), the information processing apparatus 400G performs the process of the propagation-delay-time calculating routine (Step S420) for each specified time (YES in Step S428), converts a propagation delay time into a moisture amount (Step S421), and displays the moisture amount on the display apparatus 500 or transmits the moisture amount to a server (not illustrated) (Step S429). Note that, when a moisture amount is measured in the case in which the pair of probes 201 and 202 is in the stable state of being placed, the processes of chronologically recording the moisture amounts in the memory 417 (Step S422) and chronologically displaying a history of the moisture amounts on the display apparatus 500 in the form of a graph (Step S423), which are performed in the case in which the pair of probes 201 and 202 is in the state during the insertion-and-removal period of time, do not necessarily have to be performed.

### 6-5. Twelfth Modification

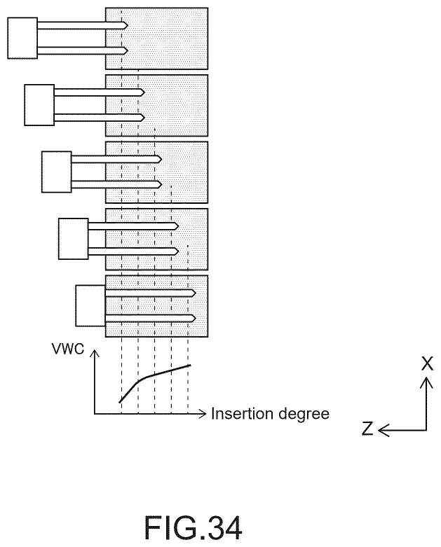

FIG. 34 illustrates a twelfth modification.

It is conceivable that a vertical hole could be dug in the medium M in, for example, a fruit farm using an excavator such as a shovel, and the probes 201 and 202 could be inserted into the medium M from an inner circumferential wall surface of the vertical hole. In this case, the axial direction Z of the probes 201 and 202 (that is, a direction of depth upon insertion) is not a vertical direction (a longitudinal direction), but is a horizontal direction (a lateral direction). In this case, a graph that has a vertical axis representing a history of moisture amounts, and a horizontal axis representing an insertion degree (a depth of antennas) may be displayed on the display apparatus 500. The axial direction Z of the probes 201 and 202 (that is, a direction of depth upon insertion), and the insertion degree (the depth of antennas) in the graph both extend on the horizontal axis in the horizontal direction (the lateral direction). This enables a user to intuitively understand a moisture distribution in the horizontal direction (the lateral direction) easily.

### 6-6. Thirteenth Modification

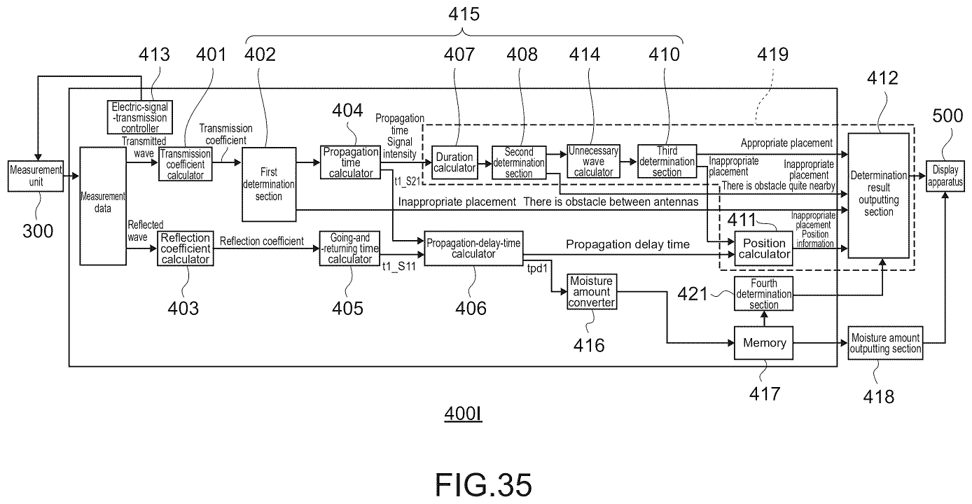

FIG. 35 illustrates a functional configuration of an information processing apparatus according to a thirteenth modification.

An information processing apparatus 400I according to the thirteenth modification has a configuration obtained by adding a fourth determination section 421 to the information processing apparatus 400G according to the second embodiment.

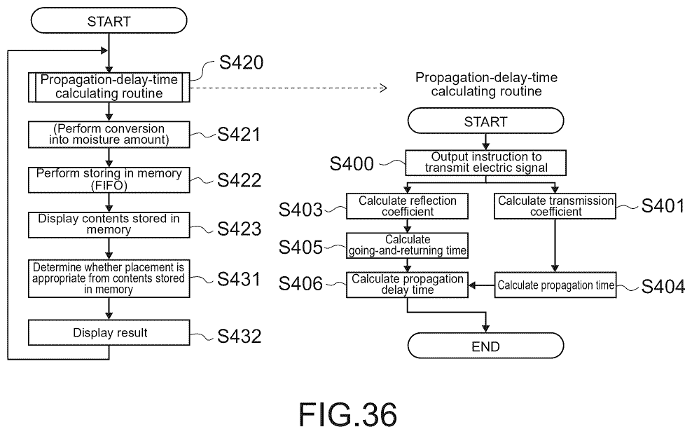

FIG. 36 illustrates a flow of an operation of the information processing apparatus.

The propagation-delay-time calculating routine (Step S420), the converting a propagation delay time into a moisture amount in a medium (Step S421), the chronologically recording the moisture amounts in the memory 417 (Step S422), and the chronologically displaying a history of the moisture amounts (Step S423) are similar to those performed in the second embodiment. On the basis of the history of the moisture amounts chronologically recorded in the memory 417, the fourth determination section 421 determines whether an object is situated in a specified range in a medium with respect to the pair of probes 201 and 202 (Step S424). For example, the fourth determination section 421 may determine whether placement is appropriate on the basis of a change in moisture amount or in propagation delay time. Specifically, when there is a rapid change, or in the case of being way beyond expectations, or when the propagation delay time of an unnecessary wave is getting close to the desired-wave propagation delay time (the NG region), the fourth determination section 421 may determine that an object is situated in the specified range in the medium with respect to the pair of probes 201 and 202. The determination result outputting section 412 outputs a result of the determination performed by the fourth determination section 421 (Step S432). When, for example, the fourth determination section 421 has determined that an object is situated in the specified range in the medium with respect to the pair of probes 201 and 202, the determination result outputting section 412 outputs, to the display apparatus 500, information (such as a message) indicating that the pair of probes 201 and 202 is placed at an inappropriate position in the medium. On the other hand, when the fourth determination section 421 has determined that no object is situated in the specified range in the medium with respect to the pair of probes 201 and 202, the determination result outputting section 412 outputs, to the display apparatus 500, information (such as a message) indicating that the pair of probes 201 and 202 is placed at an appropriate position in the medium. It is sufficient if the fourth determination section 421 at least outputs a determination result, and the fourth determination section 421 may cause the moisture amounts chronologically recorded in the memory 417 to be displayed, or does not necessarily have to cause the chronologically recorded moisture amounts to be displayed.

### 6-7. Fourteenth Modification

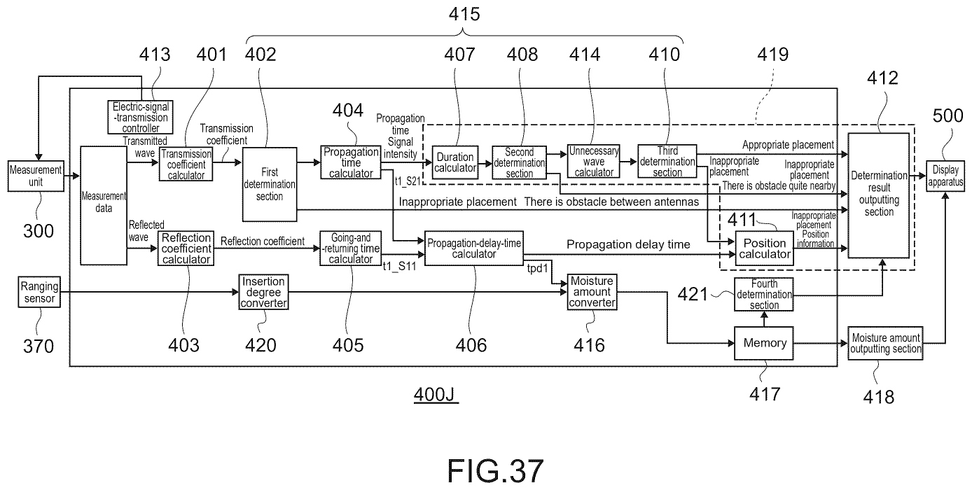

FIG. 37 illustrates a functional configuration of an information processing apparatus according to a fourteenth modification.

An information processing apparatus 400J according to the thirteenth modification has a configuration obtained by adding the fourth determination section 421 to the information processing apparatus 400H according to the eighth modification. The fourth determination section 421 operates similarly to the fourth determination section 421 in the thirteenth modification. In addition, the fourth determination section 421 determines a depth of an object that is situated in a specified range in a medium, on the basis of a depth obtained by the insertion degree converter 420. The determination result outputting section 412 may output, to the display apparatus, information that indicates a recommendation about a degree of insertion of the probes 201 and 202 into a medium, on the basis of the depth of the object and on the basis of the depth of the respective antennas 210 in the medium, the depth of the object being determined by the fourth determination section 421, the depth of the respective antennas 210 being obtained by conversion being performed by the insertion degree converter 420. For example, the determination result outputting section 412 may make a recommendation for a user about the insertion degree by outputting a message such as “more shallowly” or “more deeply” on the basis of a result of the determination, since a relationship between the insertion degree and whether placement is appropriate is known.

### 6-8. Fifteenth Modification

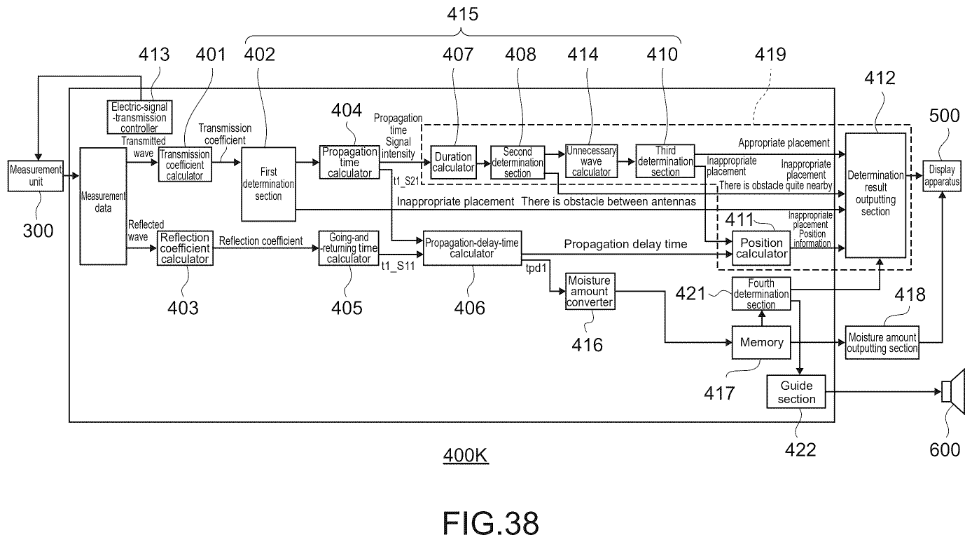

FIG. 38 illustrates a functional configuration of an information processing apparatus according to a fifteenth modification.

An information processing apparatus 400K according to the fifteenth modification has a configuration obtained by adding a guide section 422 to the information processing apparatus 400I according to the thirteenth modification. The guide section 422 outputs, to a sound output apparatus 600 or the display apparatus 500, information indicating that the pair of probes 201 and 202 is placed at an inappropriate position in a medium; or sound or information indicating a recommendation about a more appropriate placement. Typically, the sound output apparatus 600 is, for example, a speaker that is built in or connected to the information processing apparatus 400I such as a smartphone. The guide section 422 may provide guidance using sound or by performing display on the basis of a determination state or a determination result. Examples of the providing guidance include instructing to perform insertion more slowly when insertion is performed too fast, compared to a determination speed; instructing to change the placement position when an object is situated nearby; making an announcement to change modes; and instructing to follow the mode (such as instructing not to perform movement when the mode is changed to the normal mode, or instructing to perform slow movement when the mode is changed to the high-speed mode).

### 6-9. Sixteenth Modification

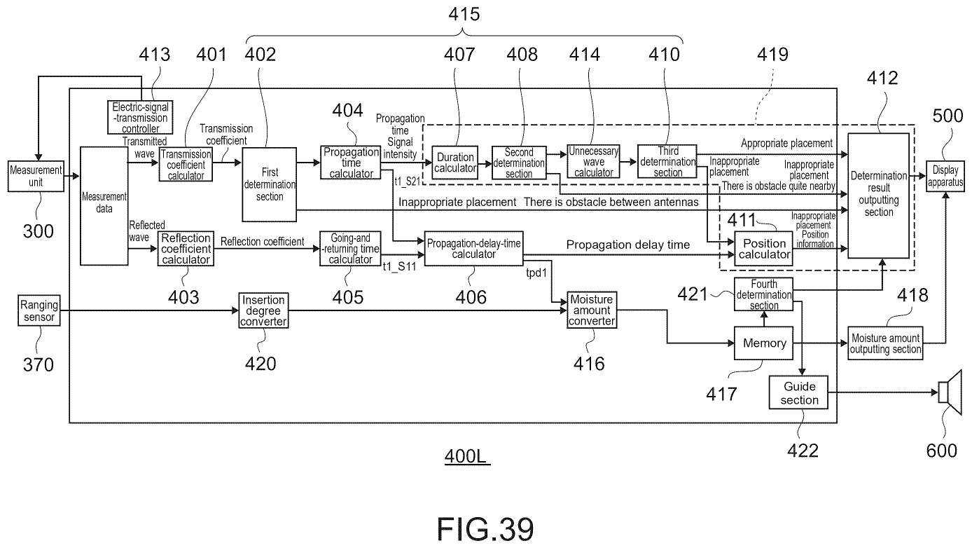

FIG. 39 illustrates a functional configuration of an information processing apparatus according to a sixteenth modification.

An information processing apparatus 400L according to the sixteenth modification has a configuration obtained by adding the guide section 422 to the information processing apparatus 400J according to the thirteenth modification. In other words, the information processing apparatus 400L according to the sixteenth modification includes all of the plurality of modes in the eleventh modification (FIG. 33), the insertion degree converter 420 and the ranging sensor 370 in the eighth modification (FIG. 28), the fourth determination section 421 in the fourteenth modification (FIG. 37), and the guide section 422 in the fifteenth modification (FIG. 38). The guide section 422 operates similarly to the guide section 422 in the fifteenth modification. In addition, the guide section 422 may output, to the sound output apparatus 600 or the display apparatus 500, sound or information used to provide guidance or make a recommendation about a degree of insertion of the probes 201 and 202 into a medium.

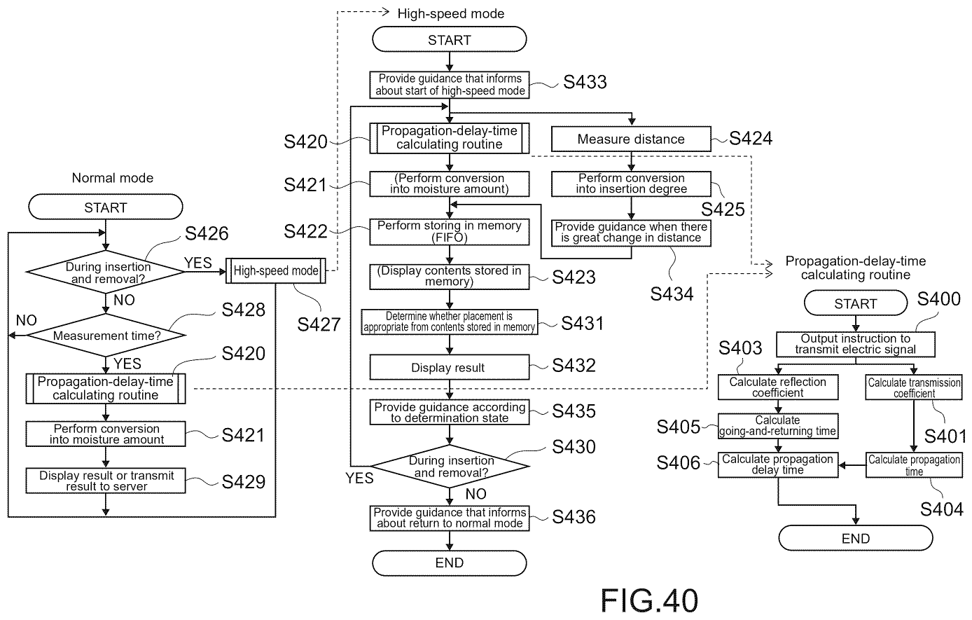

FIG. 40 illustrates a flow of an operation of the information processing apparatus.

The operation flow illustrated in FIG. 40 has a configuration obtained by adding the processes of Steps S433 to S436 to a combination of the operation flow in the eighth modification (FIG. 29), the operation flow in the eleventh modification (FIG. 33) and the operation flow in the fourteenth modification (FIG. 36). When the high-speed mode is started (Step S427), the guide section 422 outputs (Step S433) guidance informing about the start of the high-speed mode. When there is a great change in a depth of the respective antennas 210 in the medium M (a degree of insertion of the probes 201 and 202) (Step S425), the guide section 422 outputs (Step S434) guidance used to instruct to insert the probes 201 and 202 more slowly if the insertion is performed too fast, compared to a measurement speed. The guide section 422 outputs (Step S435) guidance depending on a result of the determination (Step S432) performed by the fourth determination section 421 with respect to whether an object is situated, that is, guidance used to instruct to change a placement position if an object is situated nearby. When it has been determined that the probes 201 and 202 are not in the state during the insertion-and-removal period of time but in the stable state of being placed (NO in Step S430), the guide section 422 outputs (Step S436) guidance informing about the return to the normal mode from the high-speed mode.

### 7. Conclusion

According to the second embodiment and the respective modifications, a moisture sensor having a high spatial resolution is used to continuously measure a moisture amount (a permittivity and a delay time) during insertion into and removal from a medium, and a result of the measurement is continuously displayed. This results in knowing whether the placement position is appropriate from temporary characteristics, and in knowing a moisture distribution in an insertion direction. Further, the measurement of an insertion degree using a ranging sensor makes it possible to more accurately show a relationship between the insertion degree (the depth) and the moisture amount upon measuring a moisture distribution. Furthermore, not only a propagation delay time at a peak of a desired wave (a direct wave) but also a propagation delay time at another peak (an unnecessary wave) is obtained to be displayed at the same time as the propagation delay time at the peak of the desired wave. This results in more specifically knowing whether the placement position is appropriate (for example, about an impact of an object) upon determining whether placement is appropriate. Further, the provision of a plurality of measurement modes makes it possible to perform an optimal measurement according to a state, such as performing measurement at the high-speed mode during insertion and removal, and thus to deal with a plurality of states using one type of sensor. Further, the insertion direction is not limited to a depth direction, and may be, for example, a horizontal direction. This results in knowing a moisture distribution in the horizontal direction. Furthermore, upon determining whether placement is appropriate, it is determined whether placement is appropriate on the basis of a change in moisture or a moisture distribution, or an instruction to perform insertion is given to a user. This enables the user to perform determination subjectively, not objectively. Moreover, the provision of guidance according to a measurement state or a measurement result enables a user to easily recognize a state.

Note that the present disclosure may relate to “Zero Hunger” and “Clean Water and Sanitation”, which are the Sustainable Development Goals 2 and 6, respectively, from among the Sustainable Development Goals (SDGs) established in the United Nations Summit in 2015.

The present disclosure makes it possible to measure a moisture amount in soil with a high degree of accuracy. Water spraying on crops is controlled according to a result of measurement performed by a moisture sensor, and this makes it possible to use water more efficiently.

Further, the use of the present disclosure in the field of agriculture makes it possible to reduce needless consumption of water, and wastewater in the field of agriculture, and to achieve environmentally friendly agriculture with a high degree of energy efficiency.

The present disclosure may also include the following configurations.

(1) An information processing apparatus, including:
a transmission coefficient calculator that calculates a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes; and
a determination section that determines whether an object is situated in a specified range in the medium with respect to the pair of probes, on the basis of the transmission coefficient.
   (2) The information processing apparatus according to (1), further including
a determination result outputting section that outputs, to a display apparatus, a result of the determination performed by the determination section.
   (3) The information processing apparatus according to (2), in which
when an object has been determined to be situated in the specified range in the medium with respect to the pair of probes, the determination result outputting section outputs, to the display apparatus, information indicating that the pair of probes is placed at an inappropriate position in the medium.
   (4) The information processing apparatus according to (2) or (3), in which
when an object has been determined to be situated in the specified range in the medium with respect to the pair of probes, the determination result outputting section outputs, to the display apparatus, information indicating the specified range in which the object has been determined to be situated.
   (5) The information processing apparatus according to any one of (2) to (4), in which
when an object has been determined to be situated in the specified range in the medium with respect to the pair of probes, the determination result outputting section outputs, to the display apparatus, information indicating a recommendation to place the pair of probes outside of the specified range in the medium.
   (6) The information processing apparatus according to (4) or (5), in which
the information indicating the specified range includes an image that shows the specified range.
   (7) The information processing apparatus according to any one of (1) to (6), further including
an electric-signal-transmission controller that outputs an instruction to the transmitter when a specified trigger occurs, the instruction being used to cause the transmitter to transmit the electric signal.
   (8) The information processing apparatus according to (7), in which
the specified trigger includes a timing at which the pair of probes is placed, an unchangeable periodic timing, a changeable periodic timing, and/or a change in weather.
   (9) The information processing apparatus according to any one of (1) to (8), in which
the determination section determines whether an object is situated at a specified position in the medium with respect to each of at least the two pairs of probes.
   (10) The information processing apparatus according to any one of (1) to (9), in which
the determination section includes a first determination section,
when a signal intensity at a peak for the transmission coefficient that is reached when a value of t is closest to zero exhibits a maximum value in a propagation-time range of between tA and tB, the first determination section determines that no object is situated in a first range that corresponds to the region situated between the probes of the pair of probes, in which tA represents a propagation time of propagation through a first medium that is included in the medium, and tB represents a propagation time of propagation through a second medium that is included in the medium and different from the first medium, and
when the signal intensity does not exhibit the maximum value, the first determination section determines that an object is situated in the first range.
   (11) The information processing apparatus according to any one of (1) to (10), further including
a propagation time calculator that
   calculates a propagation time and a signal intensity of the transmitted wave, and
   calculates a signal intensity A and a time t_{1m_S21} at a peak of a desired wave, in which
the determination section includes
   a duration calculator that calculates a duration F of a wave when the wave has a specified signal intensity that is less than the signal intensity A at the peak of the desired wave, and
   a second determination section that
      determines, when the duration F is greater than or equal to a first threshold, that an object is situated in a second range extending from the pair of probes, and
      determines, when the duration F is less than the first threshold, that no object is situated in the second range extending from the pair of probes.
         (12) The information processing apparatus according to any one of (1) to (11), further including
a propagation time calculator that
   calculates a propagation time and a signal intensity of the transmitted wave, and
   calculates a signal intensity A and a time t_{1m_S21} at a peak of a desired wave, in which
the determination section includes
   an unnecessary wave calculator that calculates a time difference Δt between the time t_{1m_S21} at the peak of the desired wave and a time t_{2m_S21} at a peak of an unnecessary wave by subtracting the time t_{1m_S21} from the time t_{2m_S21}, the peak of the unnecessary wave being a next peak that is reached after the time t_{1m_S21} at the peak of the desired wave, and
   a third determination section that
      determines, when Δt is greater than or equal to a second threshold, that no object is situated in a third range that is larger than the second range, and
      determines, when Δt is less than the second threshold, that an object is situated in the third range.
         (13) The information processing apparatus according to (12), in which
the unnecessary wave calculator calculates a signal intensity B of the unnecessary wave that is obtained when +Δt period of time has elapsed since the time t_{2m_S21} at the peak of the unnecessary wave,
when a value obtained by subtracting a value of the signal intensity B from a value of the signal intensity A is greater than or equal to a third threshold, the third determination section determines that no object is situated in the third range, and
when the value obtained by subtracting the value of the signal intensity B from the value of the signal intensity A is less than the third threshold, the third determination section determines that an object is situated in the third range.
   (14) The information processing apparatus according to (12) or (13), further including:
a reflection coefficient calculator that calculates a reflection coefficient from a time waveform of a reflected wave that is received by the receiver, the reflected wave being obtained by the incident wave being reflected off the one of the pair of probes;
a going-and-returning time calculator that
   calculates a going-and-returning time and a signal intensity of the reflected wave, and
   calculates a signal intensity and a time t_{1m_S11} at a peak of the reflected wave;
a propagation-delay-time calculator that calculates a propagation delay time t_{pd1} that is a difference between the time t_{1m_S21} at the peak of the desired wave and the time t_{1m_S11} at the peak of the reflected wave, the propagation delay time t_{pd1} being obtained by subtracting the time t_{1m_S11} from the time t_{1m_S21}; and
a position calculator that calculates, when the third determination section has determined that an object is situated in the third range, a collection of pieces of position information regarding positions at which the object is likely to be situated, the calculation being performed on the basis of the time difference, a position of a pair of the antennas, relative permittivity of the medium, a light speed, a distance between the probes of the pair of probes, and the propagation delay time tpdl.
   (15) The information processing apparatus according to any one of (1) to (14), further including
a propagation time calculator that
   calculates a propagation time and a signal intensity of the transmitted wave, and
   calculates a signal intensity A and a time t_{1m_S21} at a peak of a desired wave;
a reflection coefficient calculator that calculates a reflection coefficient from a time waveform of a reflected wave that is received by the receiver, the reflected wave being obtained by the incident wave being reflected off the one of the pair of probes;
a going-and-returning time calculator that
   calculates a going-and-returning time and a signal intensity of the reflected wave, and
   calculates a signal intensity and a time t_{1m_S11} at a peak of the reflected wave;
a propagation-delay-time calculator that calculates a propagation delay time t_{pd1} that is a difference between the time t_{1m_S21} at the peak of the desired wave and the time t_{1m_S11} at the peak of the reflected wave, the propagation delay time t_{pd1} being obtained by subtracting the time t_{1m_S11} from the time t_{1m_S21}l;
a moisture amount converter that
   converts the propagation delay time t_{pd1} into a moisture amount in the medium using a coefficient that represents a relationship between the moisture amount and the propagation delay time, and
   chronologically records the obtained moisture amounts in a memory; and
a moisture amount outputting section that chronologically displays, on a display apparatus, a history of the moisture amounts chronologically recorded in the memory.
   (16) The information processing apparatus according to (15), in which
each of the probes of the pair of probes includes a first end and a second end, each first end being connected to the transmitter or the receiver, each second end being spaced from a corresponding one of the first ends in an axial direction that is orthogonal to a spacing direction in which the respective antennas of a pair of the respective antennas are spaced from each other, and
each of the respective antennas is provided to a corresponding one of the pair of probes to be spaced from a corresponding one of the first ends in the axis direction.
   (17) The information processing apparatus according to (16), in which
each of the respective antennas is provided to a corresponding one of the second ends of the pair of probes.
   (18) The information processing apparatus according to (16) or (17), in which
the moisture amount converter
   continuously obtains the moisture amount in the medium for an insertion-and-removal period of time for which a depth of the respective antennas in the medium keeps on being dynamically changed by the pair of probes being inserted into and removed from the medium in the axial direction, the insertion being performed starting from the side of the second ends, and
   chronologically records, in the memory, the moisture amounts obtained for the insertion-and-removal period of time, and
the moisture amount outputting section chronologically displays, on the display apparatus, the moisture amounts obtained for the insertion-and-removal period of time and chronologically recorded in the memory.
   (19) The information processing apparatus according to (18), in which
the moisture amount outputting section displays, on the display apparatus, a graph having two axes that respectively represent the history of the moisture amounts chronologically recorded in the memory and an elapsed time corresponding to the chronological order for the recording, or that respectively represent the history of the moisture amounts chronologically recorded in the memory and the chronologically changed depth.
   (20) The information processing apparatus according to (18) or (19), further including
an insertion degree converter that converts, into the depth of the respective antennas in the medium, a distance that is measured by a ranging sensor and dynamically changed according to a degree of insertion of the pair of probes, the ranging sensor measuring a distance to a surface of the medium from the ranging sensor, in which
the moisture amount outputting section displays, on the display apparatus, a graph having two axes that respectively represent the history of the moisture amounts chronologically recorded in the memory and the chronologically changed depth.
   (21) The information processing apparatus according to any one of (18) to (20), in which
the moisture amount outputting section displays, on the display apparatus, information that indicates a depth of an object situated in the medium, the depth of the object being estimated on the basis of a change in the moisture amount relative to the depth of the respective antennas in the medium.
   (22) The information processing apparatus according to (19) or (20), in which
instead of the history of the moisture amounts, the moisture amount outputting section displays, on the graph, a history of desired-wave propagation delay times respectively corresponding to the moisture amounts, the desired-wave propagation delay time being a chronologically changed propagation delay time of the desired wave.
   (23) The information processing apparatus according to (22), in which
the moisture amount outputting section further displays a history of propagation delay times of an unnecessary wave on the graph.
   (24) The information processing apparatus according to (23), in which
the moisture amount outputting section further displays, on the graph, a range in which calculation of the desired-wave propagation delay time is likely to be affected by an object being situated in the specified range in the medium with respect to the pair of probes, the range being determined using the desired-wave propagation delay time as a reference.
   (25) The information processing apparatus according to any one of (15) to (24), in which
the moisture amount converter
   measures the moisture amount frequently during insertion and removal of the pair of probes, and
   measures the moisture amount less frequently when the pair of probes is in a non-dynamic state, compared to the case in which the frequent measurement is performed.
      (26) The information processing apparatus according to any one of (15) to (25), further including:
a fourth determination section that
   determines whether an object is situated in the specified range in the medium with respect to the pair of probes, on the basis of the history of the moisture amounts chronologically recorded in the memory or on the basis of a history of the propagation delay times, and
   determines a depth of the object when the object has been determined to be situated in the specified range in the medium with respect to the pair of probes; and
a determination result outputting section that outputs information to the display apparatus when the fourth determination section has determined that an object is situated in the specified range in the medium with respect to the pair of probes, the information indicating that the pair of probes is placed at an inappropriate position in the medium.
   (27) The information processing apparatus according to (26), further including:
a ranging sensor that measures a distance to a surface of the medium from the ranging sensor; and
an insertion degree converter that converts, into a depth of the respective antennas in the medium, the distance measured by the ranging sensor and dynamically changed according to a degree of insertion of the pair of probes, in which
the determination result outputting section outputs, to the display apparatus, information that indicates a recommendation about the degree of insertion of the pair of probes into the medium, on the basis of the depth of the object and on the basis of the depth of the respective antennas in the medium, the depth of the object being determined by the fourth determination section, the depth of the respective antennas being obtained by the conversion being performed by the insertion degree converter.
   (28) The information processing apparatus according to (27), further including
a guide section that outputs, to a sound output apparatus, sound that indicates the information indicating that the pair of probes is placed at an inappropriate position in the medium and/or the information indicating the recommendation about the degree of insertion of the pair of probes into the medium.
   (29) An information processing apparatus, including:
a transmission coefficient calculator that calculates a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes;
a propagation time calculator that
   calculates a propagation time and a signal intensity of the transmitted wave, and
   calculates a signal intensity A and a time t_{1m_S21} at a peak of a desired wave;
a reflection coefficient calculator that calculates a reflection coefficient from a time waveform of a reflected wave that is received by the receiver, the reflected wave being obtained by the incident wave being reflected off the one of the pair of probes;
a going-and-returning time calculator that
   calculates a going-and-returning time and a signal intensity of the reflected wave, and
   calculates a signal intensity and a time t_{1m_S11} at a peak of the reflected wave;
a propagation-delay-time calculator that calculates a propagation delay time t_{pd1} that is a difference between the time t_{1m_S21} at the peak of the desired wave and the time t_{1m_S11} at the peak of the reflected wave, the propagation delay time t_{pd1} being obtained by subtracting the time t_{1m_S11} from the time t_{1m_S21}l;
a moisture amount converter that
   converts the propagation delay time t_{pd1} into a moisture amount in the medium using a coefficient that represents a relationship between the moisture amount and the propagation delay time, and
   chronologically records the obtained moisture amounts in a memory; and
a moisture amount outputting section that chronologically displays, on a display apparatus, a history of the moisture amounts chronologically recorded in the memory.
   (30) An information processing method, including:
calculating a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes; and
determining whether an object is situated in a specified range in the medium with respect to the pair of probes, on the basis of the transmission coefficient.
   (31) An information processing method, including:
calculating a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes;
calculating a propagation time and a signal intensity of the transmitted wave;
calculating a signal intensity A and a time t_{1m_S21} at a peak of a desired wave;
calculating a reflection coefficient from a time waveform of a reflected wave that is received by the receiver, the reflected wave being obtained by the incident wave being reflected off the one of the pair of probes;
calculating a going-and-returning time and a signal intensity of the reflected wave;
calculating a signal intensity and a time t_{1m_S11} at a peak of the reflected wave;
calculating a propagation delay time t_{pd1} that is a difference between the time t_{1m_S21} at the peak of the desired wave and the time t_{1m_S11} at the peak of the reflected wave, the propagation delay time t_{pd1} being obtained by subtracting the time t_{1m_S11} from the time t_{1m_S21};
converting the propagation delay time t_{pd1} into a moisture amount in the medium using a coefficient that represents a relationship between the moisture amount and the propagation delay time;
chronologically recording the obtained moisture amounts in a memory; and
chronologically displaying, on a display apparatus, a history of the moisture amounts chronologically recorded in the memory.
   (32) An information processing program that causes a processor of an information processing apparatus to operate as a transmission coefficient calculator and a determination section,
the transmission coefficient calculator calculating a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes,
the determination section determining whether an object is situated in a specified range in the medium with respect to the pair of probes, on the basis of the transmission coefficient.
   (33) An information processing program that causes a processor of an information processing apparatus to operate as a transmission coefficient calculator, a propagation time calculator, a reflection coefficient calculator, a going-and-returning time calculator, a propagation-delay-time calculator, a moisture amount converter, and a moisture amount outputting section,
the transmission coefficient calculator calculating a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes,
the propagation time calculator calculating a propagation time and a signal intensity of the transmitted wave,
the propagation time calculator calculating a signal intensity A and a time t_{1m_S21} at a peak of a desired wave,
the reflection coefficient calculator calculating a reflection coefficient from a time waveform of a reflected wave that is received by the receiver, the reflected wave being obtained by the incident wave being reflected off the one of the pair of probes,
the going-and-returning time calculator calculating a going-and-returning time and a signal intensity of the reflected wave,
the going-and-returning time calculator calculating a signal intensity and a time t_{1m_S11} at a peak of the reflected wave,
the propagation-delay-time calculator calculating a propagation delay time t_{pd1} that is a difference between the time t_{1m_S21} at the peak of the desired wave and the time t_{1m_S11} at the peak of the reflected wave, the propagation delay time t_{pd1} being obtained by subtracting the time t_{1m_S11} from the time t_{1m_S21}l,
the moisture amount converter converting the propagation delay time t_{pd1} into a moisture amount in the medium using a coefficient that represents a relationship between the moisture amount and the propagation delay time,
the moisture amount converter chronologically recording the obtained moisture amounts in a memory,
the moisture amount outputting section chronologically displaying, on a display apparatus, a history of the moisture amounts chronologically recorded in the memory.
   (34) A non-transitory computer-readable recording medium that records therein an information processing program that causes a processor of an information processing apparatus to operate as a transmission coefficient calculator and a determination section,
the transmission coefficient calculator calculating a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes,
the determination section determining whether an object is situated in a specified range in the medium with respect to the pair of probes, on the basis of the transmission coefficient.
   (35) A non-transitory computer-readable recording medium that records therein an information processing program that causes a processor of an information processing apparatus to operate as a transmission coefficient calculator, a propagation time calculator, a reflection coefficient calculator, a going-and-returning time calculator, a propagation-delay-time calculator, a moisture amount converter, and a moisture amount outputting section,
the transmission coefficient calculator calculating a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes,
the propagation time calculator calculating a propagation time and a signal intensity of the transmitted wave,
the propagation time calculator calculating a signal intensity A and a time t_{1m_S21} at a peak of a desired wave,
the reflection coefficient calculator calculating a reflection coefficient from a time waveform of a reflected wave that is received by the receiver, the reflected wave being obtained by the incident wave being reflected off the one of the pair of probes,
the going-and-returning time calculator calculating a going-and-returning time and a signal intensity of the reflected wave,
the going-and-returning time calculator calculating a signal intensity and a time t_{1m_S11} at a peak of the reflected wave,
the propagation-delay-time calculator calculating a propagation delay time t_{pd1} that is a difference between the time t_{1m_S21} at the peak of the desired wave and the time t_{1m_S11} at the peak of the reflected wave, the propagation delay time t_{pd1} being obtained by subtracting the time t_{1m_S11} from the time t_{1m_S21}l,
the moisture amount converter converting the propagation delay time t_{pd1} into a moisture amount in the medium using a coefficient that represents a relationship between the moisture amount and the propagation delay time,
the moisture amount converter chronologically recording the obtained moisture amounts in a memory,
the moisture amount outputting section chronologically displaying, on a display apparatus, a history of the moisture amounts chronologically recorded in the memory.

The embodiments and the modifications of the present technology have been described above. Of course the present technology is not limited to the embodiments described above, and various modifications may be made thereto without departing from the scope of the present technology.

### REFERENCE SIGNS LIST

100 measurement apparatus
110 sensor apparatus
200 sensor head
201 probe
202 probe
210 antenna
300 measurement unit
308 cable and/or wire on substrate
310 directional coupler
320 transmitter
330 incident wave receiver
340 reflected wave receiver
350 transmitted wave receiver
360 communication section
400 information processing apparatus
401 transmission coefficient calculator
402 first determination section
403 reflection coefficient calculator
404 propagation time calculator
405 going-and-returning time calculator
406 propagation-delay-time calculator
407 duration calculator
408 second determination section
409 signal
410 third determination section
411 position calculator
412 determination result outputting section
413 electric-signal-transmission controller
414 unnecessary wave calculator
415 determination section
500 display apparatus

1. An information processing apparatus, comprising:
a transmission coefficient calculator that calculates a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes; and
a determination section that determines whether an object is situated in a specified range in the medium with respect to the pair of probes, on a basis of the transmission coefficient.

2. The information processing apparatus according to claim 1, further comprising
a determination result outputting section that outputs, to a display apparatus, a result of the determination performed by the determination section.

3. The information processing apparatus according to claim 2, wherein
when an object has been determined to be situated in the specified range in the medium with respect to the pair of probes, the determination result outputting section outputs, to the display apparatus, information indicating that the pair of probes is placed at an inappropriate position in the medium.

4. The information processing apparatus according to claim 2, wherein
when an object has been determined to be situated in the specified range in the medium with respect to the pair of probes, the determination result outputting section outputs, to the display apparatus, information indicating the specified range in which the object has been determined to be situated.

5. The information processing apparatus according to claim 2, wherein
when an object has been determined to be situated in the specified range in the medium with respect to the pair of probes, the determination result outputting section outputs, to the display apparatus, information indicating a recommendation to place the pair of probes outside of the specified range in the medium.

6. The information processing apparatus according to claim 4, wherein
the information indicating the specified range includes an image that shows the specified range.

7. The information processing apparatus according to claim 1, further comprising
an electric-signal-transmission controller that outputs an instruction to the transmitter when a specified trigger occurs, the instruction being used to cause the transmitter to transmit the electric signal.

8. The information processing apparatus according to claim 7, wherein
the specified trigger includes a timing at which the pair of probes is placed, an unchangeable periodic timing, a changeable periodic timing, and/or a change in weather.

9. The information processing apparatus according to claim 1, wherein
the determination section determines whether an object is situated at a specified position in the medium with respect to each of at least the two pairs of probes.

10. The information processing apparatus according to claim 1, wherein
the determination section includes a first determination section,
when a signal intensity at a peak for the transmission coefficient that is reached when a value of t is closest to zero exhibits a maximum value in a propagation-time range of between tA and tB, the first determination section determines that no object is situated in a first range that corresponds to the region situated between the probes of the pair of probes, in which tA represents a propagation time of propagation through a first medium that is included in the medium, and tB represents a propagation time of propagation through a second medium that is included in the medium and different from the first medium, and
when the signal intensity does not exhibit the maximum value, the first determination section determines that an object is situated in the first range.

11. The information processing apparatus according to claim 1, further comprising
a propagation time calculator that
calculates a propagation time and a signal intensity of the transmitted wave, and
calculates a signal intensity A and a time t_{1m_S21} at a peak of a desired wave, wherein
the determination section includes
a duration calculator that calculates a duration F of a wave when the wave has a specified signal intensity that is less than the signal intensity A at the peak of the desired wave, and
a second determination section that
determines, when the duration F is greater than or equal to a first threshold, that an object is situated in a second range extending from the pair of probes, and
determines, when the duration F is less than the first threshold, that no object is situated in the second range extending from the pair of probes.

12. The information processing apparatus according to claim 1, further comprising
a propagation time calculator that
calculates a propagation time and a signal intensity of the transmitted wave, and
calculates a signal intensity A and a time t_{1m_S21} at a peak of a desired wave, wherein
the determination section includes
an unnecessary wave calculator that calculates a time difference Δt between the time t_{1m_S21} at the peak of the desired wave and a time t_{2m_S21} at a peak of an unnecessary wave by subtracting the time t_{1m_S21} from the time t_{2m_S21}, the peak of the unnecessary wave being a next peak that is reached after the time t_{1m_S21} at the peak of the desired wave, and
a third determination section that
determines, when Δt is greater than or equal to a second threshold, that no object is situated in a third range that is larger than the second range, and
determines, when Δt is less than the second threshold, that an object is situated in the third range.

13. The information processing apparatus according to claim 12, wherein
the unnecessary wave calculator calculates a signal intensity B of the unnecessary wave that is obtained when +Δt period of time has elapsed since the time t_{2m_S21} at the peak of the unnecessary wave,
when a value obtained by subtracting a value of the signal intensity B from a value of the signal intensity A is greater than or equal to a third threshold, the third determination section determines that no object is situated in the third range, and
when the value obtained by subtracting the value of the signal intensity B from the value of the signal intensity A is less than the third threshold, the third determination section determines that an object is situated in the third range.

14. The information processing apparatus according to claim 12, further comprising:
a reflection coefficient calculator that calculates a reflection coefficient from a time waveform of a reflected wave that is received by the receiver, the reflected wave being obtained by the incident wave being reflected off the one of the pair of probes;
a going-and-returning time calculator that
calculates a going-and-returning time and a signal intensity of the reflected wave, and
calculates a signal intensity and a time t_{1m_S11} at a peak of the reflected wave;
a propagation-delay-time calculator that calculates a propagation delay time t_{pd1} that is a difference between the time t_{1m_S21} at the peak of the desired wave and the time t_{1m_S11} at the peak of the reflected wave, the propagation delay time t_{pd1} being obtained by subtracting the time t_{1m_S11} from the time t_{1m_S21}; and
a position calculator that calculates, when the third determination section has determined that an object is situated in the third range, a collection of pieces of position information regarding positions at which the object is likely to be situated, the calculation being performed on a basis of the time difference, a position of a pair of the antennas, relative permittivity of the medium, a light speed, a distance between the probes of the pair of probes, and the propagation delay time t_{pd1}.

15. The information processing apparatus according to claim 1, further comprising
a propagation time calculator that
calculates a propagation time and a signal intensity of the transmitted wave, and
calculates a signal intensity A and a time t_{1m_S21} at a peak of a desired wave;
a reflection coefficient calculator that calculates a reflection coefficient from a time waveform of a reflected wave that is received by the receiver, the reflected wave being obtained by the incident wave being reflected off the one of the pair of probes;
a going-and-returning time calculator that
calculates a going-and-returning time and a signal intensity of the reflected wave, and
calculates a signal intensity and a time t_{1m_S11} at a peak of the reflected wave;
a propagation-delay-time calculator that calculates a propagation delay time t_{pd1} that is a difference between the time t_{1m_S21} at the peak of the desired wave and the time t_{1m_S11} at the peak of the reflected wave, the propagation delay time t_{pd1} being obtained by subtracting the time t_{1m_S11} from the time t_{1m_S21};
a moisture amount converter that
converts the propagation delay time t_{pd1} into a moisture amount in the medium using a coefficient that represents a relationship between the moisture amount and the propagation delay time, and
chronologically records the obtained moisture amounts in a memory; and
a moisture amount outputting section that chronologically displays, on a display apparatus, a history of the moisture amounts chronologically recorded in the memory.

16. The information processing apparatus according to claim 15, wherein
each of the probes of the pair of probes includes a first end and a second end, each first end being connected to the transmitter or the receiver, each second end being spaced from a corresponding one of the first ends in an axial direction that is orthogonal to a spacing direction in which the respective antennas of a pair of the respective antennas are spaced from each other, and
each of the respective antennas is provided to a corresponding one of the pair of probes to be spaced from a corresponding one of the first ends in the axis direction.

17. The information processing apparatus according to claim 16, wherein
each of the respective antennas is provided to a corresponding one of the second ends of the pair of probes.

18. The information processing apparatus according to claim 16, wherein
the moisture amount converter
continuously obtains the moisture amount in the medium for an insertion-and-removal period of time for which a depth of the respective antennas in the medium keeps on being dynamically changed by the pair of probes being inserted into and removed from the medium in the axial direction, the insertion being performed starting from the side of the second ends, and
chronologically records, in the memory, the moisture amounts obtained for the insertion-and-removal period of time, and
the moisture amount outputting section chronologically displays, on the display apparatus, the moisture amounts obtained for the insertion-and-removal period of time and chronologically recorded in the memory.

19. The information processing apparatus according to claim 18, wherein
the moisture amount outputting section displays, on the display apparatus, a graph having two axes that respectively represent the history of the moisture amounts chronologically recorded in the memory and an elapsed time corresponding to the chronological order for the recording, or that respectively represent the history of the moisture amounts chronologically recorded in the memory and the chronologically changed depth.

20. The information processing apparatus according to claim 18, further comprising
an insertion degree converter that converts, into the depth of the respective antennas in the medium, a distance that is measured by a ranging sensor and dynamically changed according to a degree of insertion of the pair of probes, the ranging sensor measuring a distance to a surface of the medium from the ranging sensor, wherein
the moisture amount outputting section displays, on the display apparatus, a graph having two axes that respectively represent the history of the moisture amounts chronologically recorded in the memory and the chronologically changed depth.

21. The information processing apparatus according to claim 18, wherein
the moisture amount outputting section displays, on the display apparatus, information that indicates a depth of an object situated in the medium, the depth of the object being estimated on a basis of a change in the moisture amount relative to the depth of the respective antennas in the medium.

22. The information processing apparatus according to claim 19, wherein
instead of the history of the moisture amounts, the moisture amount outputting section displays, on the graph, a history of desired-wave propagation delay times respectively corresponding to the moisture amounts, the desired-wave propagation delay time being a chronologically changed propagation delay time of the desired wave.

23. The information processing apparatus according to claim 22, wherein
the moisture amount outputting section further displays a history of propagation delay times of an unnecessary wave on the graph.

24. The information processing apparatus according to claim 23, wherein
the moisture amount outputting section further displays, on the graph, a range in which calculation of the desired-wave propagation delay time is likely to be affected by an object being situated in the specified range in the medium with respect to the pair of probes, the range being determined using the desired-wave propagation delay time as a reference.

25. The information processing apparatus according to claim 15, wherein
the moisture amount converter
measures the moisture amount frequently during insertion and removal of the pair of probes, and
measures the moisture amount less frequently when the pair of probes is in a non-dynamic state, compared to the case in which the frequent measurement is performed.

26. The information processing apparatus according to claim 15, further comprising:
a fourth determination section that
determines whether an object is situated in the specified range in the medium with respect to the pair of probes, on a basis of the history of the moisture amounts chronologically recorded in the memory or on a basis of a history of the propagation delay times, and
determines a depth of the object when the object has been determined to be situated in the specified range in the medium with respect to the pair of probes; and
a determination result outputting section that outputs information to the display apparatus when the fourth determination section has determined that an object is situated in the specified range in the medium with respect to the pair of probes, the information indicating that the pair of probes is placed at an inappropriate position in the medium.

27. The information processing apparatus according to claim 26, further comprising:
a ranging sensor that measures a distance to a surface of the medium from the ranging sensor; and
an insertion degree converter that converts, into a depth of the respective antennas in the medium, the distance measured by the ranging sensor and dynamically changed according to a degree of insertion of the pair of probes, wherein
the determination result outputting section outputs, to the display apparatus, information that indicates a recommendation about the degree of insertion of the pair of probes into the medium, on a basis of the depth of the object and on a basis of the depth of the respective antennas in the medium, the depth of the object being determined by the fourth determination section, the depth of the respective antennas being obtained by the conversion being performed by the insertion degree converter.

28. The information processing apparatus according to claim 27, further comprising
a guide section that outputs, to a sound output apparatus, sound that indicates the information indicating that the pair of probes is placed at an inappropriate position in the medium and/or the information indicating the recommendation about the degree of insertion of the pair of probes into the medium.

29. An information processing apparatus, comprising:
a transmission coefficient calculator that calculates a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes;
a propagation time calculator that
calculates a propagation time and a signal intensity of the transmitted wave, and
calculates a signal intensity A and a time t_{1m_S21} at a peak of a desired wave;
a reflection coefficient calculator that calculates a reflection coefficient from a time waveform of a reflected wave that is received by the receiver, the reflected wave being obtained by the incident wave being reflected off the one of the pair of probes;
a going-and-returning time calculator that
calculates a going-and-returning time and a signal intensity of the reflected wave, and
calculates a signal intensity and a time t_{1m_S11} at a peak of the reflected wave;
a propagation-delay-time calculator that calculates a propagation delay time t_{pd1} that is a difference between the time t_{1m_S21} at the peak of the desired wave and the time t_{1m_S11} at the peak of the reflected wave, the propagation delay time t_{pd1} being obtained by subtracting the time t_{1m_S11} from the time t_{1m_S21};
a moisture amount converter that
converts the propagation delay time t_{pd1} into a moisture amount in the medium using a coefficient that represents a relationship between the moisture amount and the propagation delay time, and
chronologically records the obtained moisture amounts in a memory; and
a moisture amount outputting section that chronologically displays, on a display apparatus, a history of the moisture amounts chronologically recorded in the memory.

30. An information processing method, comprising:
calculating a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes; and
determining whether an object is situated in a specified range in the medium with respect to the pair of probes, on a basis of the transmission coefficient.

31. An information processing method, comprising:
calculating a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes;
calculating a propagation time and a signal intensity of the transmitted wave;
calculating a signal intensity A and a time t_{1m_S21} at a peak of a desired wave;
calculating a reflection coefficient from a time waveform of a reflected wave that is received by the receiver, the reflected wave being obtained by the incident wave being reflected off the one of the pair of probes;
calculating a going-and-returning time and a signal intensity of the reflected wave;
calculating a signal intensity and a time t_{1m_S11} at a peak of the reflected wave;
calculating a propagation delay time t_{pd1} that is a difference between the time t_{1m_S21} at the peak of the desired wave and the time t_{1m_S11} at the peak of the reflected wave, the propagation delay time t_{pd1} being obtained by subtracting the time t_{1m_S11} from the time t_{1m_S21};
converting the propagation delay time t_{pd1} into a moisture amount in the medium using a coefficient that represents a relationship between the moisture amount and the propagation delay time;
chronologically recording the obtained moisture amounts in a memory; and
chronologically displaying, on a display apparatus, a history of the moisture amounts chronologically recorded in the memory.

32. An information processing program that causes a processor of an information processing apparatus to operate as a transmission coefficient calculator and a determination section,
the transmission coefficient calculator calculating a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes,
the determination section determining whether an object is situated in a specified range in the medium with respect to the pair of probes, on a basis of the transmission coefficient.

33. An information processing program that causes a processor of an information processing apparatus to operate as a transmission coefficient calculator, a propagation time calculator, a reflection coefficient calculator, a going-and-returning time calculator, a propagation-delay-time calculator, a moisture amount converter, and a moisture amount outputting section,
the transmission coefficient calculator calculating a transmission coefficient from a time waveform of a transmitted wave that is transmitted through a region in a medium and received by a receiver that receives the transmitted wave, the transmitted wave being from among incident waves that are transmitted by a transmitter that transmits an electric signal including the incident wave to one of a pair of probes each including an antenna, the region in the medium being situated between probes of the pair of probes,
the propagation time calculator calculating a propagation time and a signal intensity of the transmitted wave,
the propagation time calculator calculating a signal intensity A and a time t_{1m_S21} at a peak of a desired wave,
the reflection coefficient calculator calculating a reflection coefficient from a time waveform of a reflected wave that is received by the receiver, the reflected wave being obtained by the incident wave being reflected off the one of the pair of probes,
the going-and-returning time calculator calculating a going-and-returning time and a signal intensity of the reflected wave,
the going-and-returning time calculator calculating a signal intensity and a time t_{1m_S11} at a peak of the reflected wave,
the propagation-delay-time calculator calculating a propagation delay time t_{pd1} that is a difference between the time t_{1m_S21} at the peak of the desired wave and the time t_{1m_S11} at the peak of the reflected wave, the propagation delay time t_{pd1} being obtained by subtracting the time t_{1m_S11} from the time t_{1m_S21}l,
the moisture amount converter converting the propagation delay time t_{pd1} into a moisture amount in the medium using a coefficient that represents a relationship between the moisture amount and the propagation delay time,
the moisture amount converter chronologically recording the obtained moisture amounts in a memory,
the moisture amount outputting section chronologically displaying, on a display apparatus, a history of the moisture amounts chronologically recorded in the memory.
